(19) United States
(12) Patent Application Publication
(10) Pub. No.: US 2024/0276984 A1
(43) Pub. Date: Aug. 22, 2024

(54) ANTIBACTERIAL / ANTIVIRAL BIOCIDE APPARATUS AND METHOD OF USE THEREOF

(71) Applicants: Mark Oman, West Des Moines, IA (US); Marion L. Chiattello, Cedar Falls, IA (US)

(72) Inventors: Mark Oman, West Des Moines, IA (US); Marion L. Chiattello, Cedar Falls, IA (US)

(21) Appl. No.: 18/645,880

(22) Filed: Apr. 25, 2024

Related U.S. Application Data
(63) Continuation-in-part of application No. 18/641,922, filed on Apr. 22, 2024.
Application No. 18/641,922 is a continuation-in-part of application No. 18/638,981, filed on Apr. 18, 2024.
Application No. 18/638,981 is a continuation-in-part of application No. 18/637,668, filed on Apr. 17, 2024.
Application No. 18/637,668 is a continuation-in-part of application No. 17/869,477, filed on Jul. 20, 2022.
Application No. 17/869,477 is a continuation-in-part of application No. 15/662,119, filed on Jul. 27, 2017, now Pat. No. 11,426,343.
(60) Provisional application No. 62/488,421, filed on Apr. 21, 2017.
(60) Provisional application No. 62/368,008, filed on Jul. 28, 2016.
(60) Provisional application No. 63/567,553, filed on Mar. 20, 2024.
(60) Provisional application No. 63/569,165, filed on Mar. 24, 2024.
(60) Provisional application No. 63/633,967, filed on Apr. 15, 2024.

(51) Int. Cl. A01N 33/04; A01P 1/00; C08J 3/07; C08J 5/18

(52) U.S. Cl. CPC A01N 33/04; A01P 1/00; C08J 3/07; C08J 5/18; C08J 2379/02

(57) ABSTRACT

The invention comprises a method for generating a biocide, comprising the steps of generating a solution of polymers comprising charged amines on nitrogen containing repeating backbone monomers, the solution comprising chloride anions as counterions to greater than fifty percent of the charged amines and a pH less than four; and subsequently: (1) increasing a pH of the solution by at least one pH unit and (2) decreasing the chloride anion concentration of the solution by at least five percent to yield a reduced chloride anion solution, the reduced chloride anion solution yielding the biocide, the biocide inactivating, with physical contact for greater than two minutes, both: greater than fifty percent of a bacteria and fifty percent of a virus. Methods of increasing the pH and decreasing the chloride concentration optionally include increasing the pH, forming a solid salt, rinsing and lowering the pH and/or using an anion exchange material.

### CROSS-REFERENCES TO RELATED APPLICATIONS

This application:
is a continuation-in-part of U.S. patent application Ser. No. 18/641,922 filed Apr. 22, 2024, which is a continuation-in-part of U.S. patent application Ser. No. 18/638,981 filed Apr. 18, 2024, which is a continuation-in-part of U.S. patent application Ser. No. 18/637,668 filed Apr. 17, 2024, which:
   is a continuation-in-part of U.S. patent application Ser. No. 17/869,477 filed Jul. 20, 2022, which is a continuation of U.S. patent application Ser. No. 15/662,119 filed Jul. 27, 2017 (now U.S. Pat. No. 11,426,343), which:
      claims benefit of U.S. provisional patent application No. 62/488,421 filed Apr. 21, 2017; and
      claims benefit of U.S. provisional patent application No. 62/368,008 filed Jul. 27, 2016; and
   claims benefit of U.S. provisional patent application No. 63/567,553 filed Mar. 20, 2024;
   claims benefit of U.S. provisional patent application No. 63/569,165 filed Mar. 24, 2024; and
   claims benefit of U.S. provisional patent application No. 63/633,967 filed Apr. 15, 2024.

### BACKGROUND OF THE INVENTION

### Field of the Invention

The invention relates generally to antimicrobial/antiviral solutions/films/coatings/compositions.

### Discussion of the Prior Art

### Problem

There exists in the art a need for a forming and implementing antimicrobial/antiviral solutions, films, and/or coatings.

### SUMMARY OF THE INVENTION

The invention comprises antimicrobial/antiviral solutions, films, and coating apparatus and method of use/formation thereof.

### DESCRIPTION OF THE FIGURES

A more complete understanding of the present invention is derived by referring to the detailed description and claims when considered in connection with the Figures, wherein like reference numbers refer to similar items throughout the Figures.

FIG. 1 illustrates a method of tuning a polymer as an antimicrobial agent;

FIG. 2 illustrates tuned charged polymers and tuned counterions;

FIG. 3A and FIG. 3B each illustrate tuned cationic polymers;

FIG. 4 illustrates polymer properties/states;

FIG. 5 illustrates a set of polymers;

FIG. 6 illustrates polymer protonation tuning;

FIG. 7 illustrates polymer counterion tuning;

FIG. 8 illustrates polymer acidification/alkalization processes;

FIG. 9 illustrates acidification of a polymer;

FIG. 10 illustrates alkalization of a polymer;

FIG. 11 illustrates a first alkalization polymer tuning process;

FIG. 12 illustrates a second alkalization polymer tuning process;

FIG. 13 illustrates a third alkalization polymer tuning process;

FIG. 14 illustrates a first acidification polymer tuning process;

FIG. 15 illustrates a conjugate base polymer tuning process;

FIG. 16 illustrates a polymer slurry acid reduction process;

FIG. 17 illustrates a polymer-salt pair salt reduction process;

FIG. 18 illustrates polymer tuning with a weak acid;

FIG. 19A and FIG. 19B each illustrate polymer tuning with a weak acid and salt production;

FIG. 20, illustrates relative concentration of conjugate base tuning;

FIG. 21 illustrates first conjugate base availability determination;

FIG. 22 illustrates second conjugate base availability determination;

FIG. 23 illustrates adjusting conjugate base concentration;

FIG. 24 illustrates relative tuning of two conjugate bases;

FIG. 25A and FIG. 25B illustrate two counterion ratios;

FIG. 26 illustrates relative concentration of conjugate base tuning with three counterion types;

FIG. 27 illustrates relative concentration of conjugate base tuning with polyanionic compounds;

FIG. 28 illustrates relative tuning of multiple conjugate bases/counterions;

FIG. 29 illustrates charged chemical backbone structures;

FIG. 30A illustrates a primary amine, FIG. 30B illustrates a secondary amine, FIG. 30C illustrates a tertiary amine, and FIG. 30D illustrates a quaternary amine;

FIG. 31A and FIG. 31B illustrate deprotonated and protonated repeating units, respectively, FIG. 31C illustrates a polypropylenimine, FIG. 31D illustrates a generalized amine polymer, and FIG. 31E illustrates a nitrogen containing repeating backbone monomer of a polymer;

FIG. 32 illustrates conjugate acid formation from a base;

FIG. 33A, FIG. 33B, FIG. 33C, and FIG. 33D illustrate progressively basic to acidic forms of polyethylenimine;

FIG. 34 illustrates branched polyethylenimine;

FIG. 35 illustrates a backbone using amino acids; and

FIG. 36 illustrates a generic backbone structure.

Elements and steps in the figures are illustrated for simplicity and clarity and have not necessarily been rendered according to any particular sequence. For example, steps that are performed concurrently or in different order are illustrated in the figures to help improve understanding of embodiments of the present invention.

### DETAILED DESCRIPTION OF THE INVENTION

The invention comprises a method for generating a biocide, comprising the steps of generating a solution of polymers comprising charged amines on nitrogen containing repeating backbone monomers, the solution comprising chloride anions as counterions to greater than fifty percent of the charged amines and a pH less than four; and subsequently: (1) increasing a pH of the solution by at least one pH unit and (2) decreasing the chloride anion concentration of the solution by at least five percent to yield a reduced chloride anion solution, the reduced chloride anion solution yielding the biocide, the biocide inactivating, with physical contact for greater than two minutes, both: greater than fifty percent of a bacteria and fifty percent of a virus. Methods of increasing the pH and decreasing the chloride concentration optionally include increasing the pH, forming a solid salt, rinsing and lowering the pH and/or using an anion exchange material.

Herein, EPA refers to the United States Environmental Protection Agency and ISO stands for the International Organization for Standardization.

### Overview

Generally, methods and processes that comprise the implementation of a new polymer-based antimicrobial technology that provides safe, durable, and/or readily removable antimicrobial films capable of inactivating bacteria, viruses, and/or fungi, in real-time with protection into the future, and are durable enough to pass both the EPA (O1-1A) and PAS2424 international durability tests are described herein. The PAS stands for a publicly available specification and the PAS2424 is from the British Standards Institution.

More specifically, parameters for forming and implementing various polymers, such as cationic polymers; techniques for protonating the cationic polymers to achieving maximum charge; and formulation with various counter ions are provided herein.

The high performance and safety of products created by this invention are derived from the inventive use of charged polymers, such as cationic polymers, and their concomitant counter ions, which is starkly different than the current use of toxic small molecule antimicrobial chemicals in the art.

### Technology Attributes

Key attributes of the technology are:
1. Use of formulations/dispersions of polymers and/or small polymer particle dispersions, hereinafter referred to as cationic polymers for clarity of presentation and without loss of generality, are used as disinfectants, such as when at least partially suspended in the liquid state.
2. In a solid state the cationic polymers create a contact-active durable antimicrobial film that yields future protection against disease-causing pathogens, which is starkly different than legacy disinfectants.
3. Because the cationic polymer technology is not based on toxic germicidal chemicals, new products arise from the tuned polymers, described infra, that are safe for humans, animals, and/or the environment.
4. The cationic polymer technology is optionally implemented in antimicrobial films capable of inactivating: bacteria, viruses, and/or fungi as demonstrated in ISO 22196 testing resulting in reduction in microbial population by a factor of 10,000 or more (referred to herein as “>log-4 inactivation”) against a wide range of bacteria and fungus in ten minutes.
5. Antimicrobial films incorporating the cationic polymer technology kill both enveloped and non-enveloped viruses.
6. The cationic polymers, such as described herein, show a continuous (additive) killing action against a large population of bacteria, with inactivating >log 7 population, on a 5-day old film, as tested with ISO 22196.
7. Antimicrobial films created with the cationic polymers described herein pass both the EPA 01-1A durability test and the PAS2424 international durability test against, bacteria, virus, and fungi. The protocol of EPA 01-1A is incorporated in its herein as published at: entirety https://www.epa.gov/sites/default/files/2015-09/documents/cloroxpcol_final.pdf.
8. Unlike germicidal chemical disinfectants that quickly dissipate, the polymeric antimicrobial films reported herein provide an ongoing continuous killing action, such as for days and/or weeks.
9. The methods and processes taught herein are flexible and adaptable allowing for the creative implementation of new products, all to the benefit of humankind.

Generally, ISO 22196 is a standard that pertains to the measurement of antibacterial activity on plastics and other non-porous surfaces, which specifies a test method to evaluate the antibacterial activity of such surfaces by measuring the ability of bacteria to survive and multiply on them. Stated again, if a material undergoes ISO 22196 testing and achieves log-4 inactivation, it means that it has demonstrated a strong ability to inhibit or kill bacteria, making it suitable for applications where maintaining a hygienic environment is essential, such as in healthcare facilities, food processing areas, or public spaces.

### Antimicrobial Action

The antimicrobial cationic polymers films described herein provide antimicrobial action in at least one and optionally in all of several ways:
1. the polymer at least partially sterically blocks action of the bacteria/virus/fungus;
2. the cationic polymer attracts and then disrupts the anionic charge of a pathogens biological membrane;
3. counter ions chemically denature the membrane and can enter the pathogen's interior; and
4. the acidic nature of the disinfectant disrupts the natural pH of the pathogens' interior chemistry.

### BACKGROUND

There is an urgent worldwide need for a new antimicrobial technology that aids in mitigation of the transmission of infectious diseases.

There are two main reasons for not winning the war against disease-causing pathogens: First, despite the development of vaccines and antibiotics, the war on infectious diseases is being lost because the focus has been primarily on using medical interventions to combat disease-causing pathogens inside the body rather than helping to eradicate germs outside the body using disinfectants. This is in large part because current disinfectant technologies are limited. The disinfectants of today only kill pathogens while wet. Thus, they do not offer continuous, long-term protection. Further, to truly combat the transmission of infectious diseases, there is a need for residual coating disinfectants that can continuously and effectively inactivate disease-causing pathogens, as described herein, on surfaces, textiles, and skin/hands over an extended period, and do so with little or no toxicity. Second, presently insufficient emphasis is placed on disrupting the chain of infection. This is largely because existing technologies cannot break the chain. They only “disinfect”. They do not “protect”.

The “chain of infection” describes how diseases are transmitted from surfaces to hands, to the face, and then into the body. It is estimated that 80% of human infections occur from microbe-contaminated surfaces and that hands are the main pathway to pathogens to enter the body. One study showed that humans, on average, touch their face twenty-three times per hour. Today's hand sanitizers do not break the “chain of infection” and/or mode of transmission.

Legacy hand sanitizers are only effective while the disinfectant is drying. Once dried, they provide little protection. It is only a matter of time before hands become re-infected with disease-causing pathogens

Understanding the chain of infection narrative is important because it helps prioritize various disinfectant applications, especially in the context of residual antimicrobial films that provide longer term protection.

A residual hand sanitizer, such as using the cationic polymers described herein, is a beneficial application because hands are the major object of transmission. When contaminated surfaces are touched the transmission is two-fold: first to the individual as he or she touches their face and second to other surfaces which in turn exposes others.

Perhaps the next most important application to offer residual protection is textile clothing. This is because individual's hand touches their clothing and then they continue to carry the disease-causing germs with them, which exposes others.

Protecting surfaces, is important but in general it is limited, because there are so many surfaces to be protected. Still, protecting surfaces is important, especially key surfaces such as in healthcare settings and/or in food preparation.

Since most viruses are transmitted as an aerosol spray, a truly anti-viral face mask could significantly mitigate the transmission of viruses. The polymeric anti-viral coatings described herein are optionally and preferably used in an aerosol spray and/or a coating to achieve this.

Small molecule germicidal chemical are not capable of creating durable residual antimicrobial films. However, the cationic polymers described herein are applicable to durable applications of residual antimicrobial films.

Regulators require that for a residual claim to be made, antimicrobial films must pass in the USA the EPA (O1-1A) and internationally the PAS2424 durability protocols; the cationic polymer films described herein more than adequately meet the standards of these tests. Herein, the antiviral inactivity of a virus exceeds 10, 20, 30, 40, 50, 60, 70, 80, 90, 99, 99.9, and/or 99.99 fifty percent inactivity after a contact time of said virus with said antimicrobial film exceeding five minutes.

### Applications

Stakeholders and the EPA are asking for disinfectants that leave a residual antimicrobial coating that “kill”/block disease causing pathogens into the future.

More specifically, the EPA is seeking new disinfectant technologies to offer essentially two things. First, a residual antimicrobial action against a wide spectrum of pathogens, but particularly against viruses; and second, a new technology not based on toxic biocides and that provides residual longer-term protection on surfaces and skin.

The methods and processes taught herein meet these standards as they are based on non-toxic polymeric coatings, are applicable to coating a range of surfaces, and leave residual antimicrobial coatings. These new products significantly reduce the transmission of infectious diseases, particularly in a world where viral pandemics are a reality.

For clarity of presentation and without loss of generality, some examples include of applications of the tuned polymers, described infra, are to: (1) consumer and health care products, (2) medical applications, (3) industrial applications, and (4) agricultural applications, as further described herein.

Consumer and health care products:
1. A residual kill hand sanitizer is a preferred application for this new technology. Until now, the EPA and other regulators have not approved residual “killing” hand sanitizers due to safety and effectiveness.

Based on methods described herein, an extremely low toxicity, durable, and high killing action residual, polymer-based hand sanitizer has been produced. The viability of such a residual hand sanitizer was demonstrated with a >log-3 kill of bacteria and enveloped virus on artificial skin exposed to a 24 hour durability test as per the PAS2424 international durability protocol.
2. The tuned polymer is optionally used as a textile residual protective films on rinse additives, band-aids, and/or dryer sheets and/or is applied as an aerosol spray.
3. The tuned polymer is optionally used as a spray or wipe disinfectant that inactivates pathogens faster than traditional disinfectants and then forms a self-sanitizing antimicrobial coating on nonporous surfaces.

### Medical Applications:

Medical applications of the tuned polymer include, but are not limited to, antiviral face masks, medical device sterilant, medical device hoses, surgical handwash, wound dressings, and antimicrobial ointments, and/or reducing biofilms on implants. The highly durable and remarkably non-toxic coatings offered by this technology reduce biofilm accumulation on various implanted medical devices.

### Industrial Applications:

Industrial applications of the tuned polymer includes replacing/supplementing any existing toxic biocides, such as used in packaging and industrial fluids.

### Agricultural Applications:

Agricultural examples of applications of the tuned polymer optionally include animal skin care or antimicrobial protection in large feeding operations, which reduces healthcare acquired infections (HAI) that stem largely from bacteria and or individual illnesses due to viruses.

Generally, the continuous killing action of the antimicrobial polymeric films/compositions produced by the processes taught herein are a novel approach to acting on bacteria and also, over time, a surface can remain virtually bacteria-free, which reduces the potential for residual (self-generating) bacteria to mutate and become antibiotic-resistant.

### Tuned Polymer

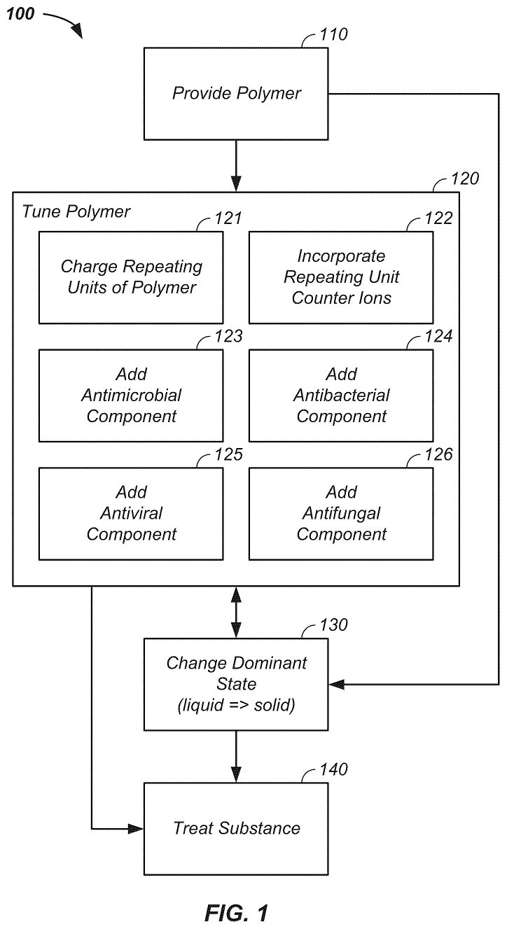

Generally, the polymers described herein are “tuned” in one or more ways as illustrated and described in FIGS. 1-20.

Referring now to FIG. 1, polymer tuning 100 is described. Generally, in a first process, a polymer is provided 110. In a second process, the polymer is tuned 120. The process of tuning a polymer is detailed infra; however, generally, repeating units of the polymer are charged 121 and/or repeating unit counter ions are incorporated 122 to, separately and/or in combination, yield an antimicrobial component 123, an antibacterial component 124, an antiviral component 125, and/or an antifungal component 126. In an optional third process, the physical state of the tuned polymer is changed 130, such as from a liquid, to a gel, to a solid or vice versa. In a fourth process, the resulting tuned polymer is optionally used to treat a substance 140 and/or to treat a material or condition. Optional and preferably methods of tuning the polymer are further described, infra. Optionally, a protein is substituted for a polymer in any embodiment herein. Optionally, any backbone chain is substituted for the polymer in any embodiment herein. For instance, any organic molecule having a chain of carbons at least 3, 5, 10, 15, 20, 100, 500, or 1000 carbons long is optionally used in place of the polymer as described herein. Optionally, a backbone molecule is used in place of the polymer 200. For instance, a chain of any combination of carbons, oxygens, and nitrogens of any length greater than 2, 5, 10, 20, or 50 atoms is used as a backbone where the backbone is optionally protonated, has counterions, and/or is tuned according to any of the approaches described herein for tuning a polymer.

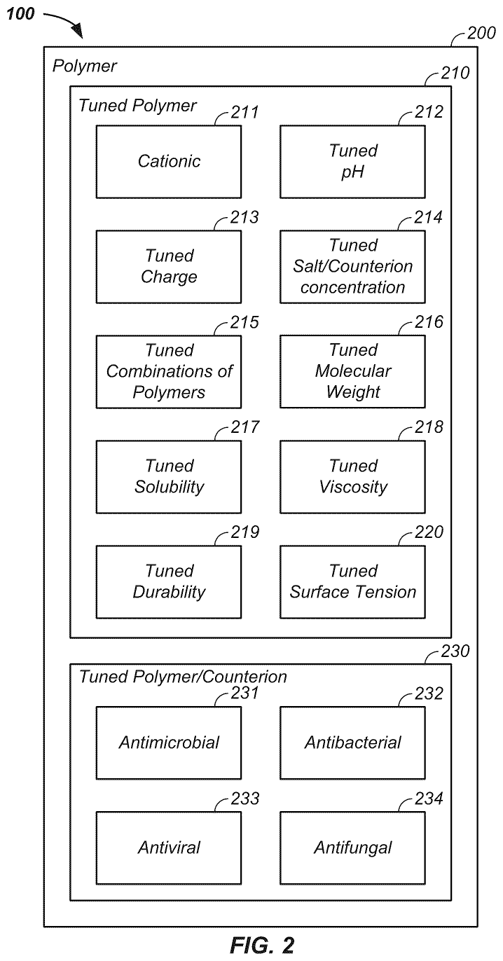

Referring now to FIG. 2, a polymer 200, such as provided in the polymer tuning 100 process, is further described.

Still referring to FIG. 2, in a first process, the polymer 200 is optionally tuned to form a tuned polymer 210 having a state of cationic, tuned pH, tuned charge, tuned salts/counterions, tuned combinations of polymers, tuned molecular weight, tuned solubility, tuned viscosity, tuned durability, and/or tuned surface tension, which are each further described, infra. Generally, any one or more of the tuning steps are optionally performed in any order and/or are performed simultaneously, as further described infra. For clarity of presentation and without loss of generality, a polymer with repeating cationic units is used as an example herein top describe the chemistry, apparatus, methods, and processes and in particular a specific polymer of polyethylenimine is used to illustrate any polymer with repeating units/monomers that are charged and/or are chargeable.

Still referring to FIG. 2, the tuned polymer 210 is optionally and preferably cationic 211 and/or has repeating cationic polymer units; however, the polymer 200 is optionally anionic and/or has repeating anionic polymer units; and/or has a functional group that is optionally subjected to a chemical reaction to form a cationic, anionic, and/or zwitterionic repeating group on repeating sections of the polymer 200.

Notably, while the polymer 200 is optionally and preferably tuned; optionally and preferably the formulation that contains the polymer is optionally tuned to any of the below described levels of the tuned polymer 210, such as in terms of viscosity, durability, surface tension, and/or in terms of effectiveness against any microbe, bacteria, virus, and/or fungus.

Still referring to FIG. 2, the polymer 200 is optionally and preferably pH tuned 212 to a desired pH, such as: in a range with a pH greater than 2, 3, 4, or 5; to a pH less than 14, 12, 10, 8, 7, or 6; in a pH range of 2-10, 3-9, 3-8, 3-7, 4-6; to a pH within any of ±0.25, 0.5, 0.75, 1.0, or 2 of any of 3, 4, 5, 6, or 7; and/or any combination of these ranges.

Still referring to FIG. 2, the polymer 200 is optionally and preferably tuned to a charge, such as in a range of 1*10⁻²⁰ to 1*10⁻¹² coulombs/molecule and more preferably in a range of 1*10⁻¹⁸ to 1*10⁻¹⁴ coulombs/molecule; coulombs abbreviated as C. Optionally and preferably, between 10 and 90 percent of the secondary amines of polyethylenimine 250 are positively charged or the same percentage of any protonatable group of the polymer 200. More preferably, greater than 15, 20, 25, or 30 percent of the nitrogens, or protonatable groups, are positively charged and/or less than 80, 70, 60, 50, or 40 percent of the protonatable groups are charged. Herein, a total cationic charge is a charge of all of the positively charged groups, which does not include anionic charge from any counterion. A preferred total cationic charge is 8.43*10⁷ Coulombs per mol plus/minus 10, 20, 30, 40, or 50 percent. Optionally and preferably, a total cationic charge used in a biocide treatment, such as in a unit of liquid dispensed and/or in a film formed is greater than 0.2, 0.4, 0.6, 0.8, 1, 1.2, 1.4, or 1.8 coulombs and/or is less than 100, 75, 50, 25, 10, or 5 coulombs, such as where a unit of fluid delivered is within 10, 25, or 50% of any of 0.1, 0.2, 0.5, 1, 2, 3, 5, or 10 mL. A preferred total cationic charge per polymer is optionally tuned, such as in a range of 8*10⁵ to 8*10⁹ coulombs/mol and more preferably in a range of 8*10⁶ to 8*10⁸ coulombs/mol. Optionally, any of the charges per molecule are optionally reduced by 10, 20, 30, 40, 50, 60, 70, 80, 90, or more percent as the total chain lengths of polymer decreases from thousands to hundreds to tens.

Still referring to FIG. 2, the polymer 200 is optionally salt/counterion tuned 214 with one or more salt anions in solution; salt cations in solution, and/or counterions, which are optionally anionic or cationic; electrostatically and/or attracted via charge to charges, such as protons on monomers of the polymer 200, as further described infra.

Still referring to FIG. 2, the polymer 200 is optionally a tuned combination of polymers 215, such as a mix of molecular weights, polymer types, and/or types of counterions associated with each individual polymer or set of polymers. Further, the tuned combination of polymers 215 optionally contains a polymer or a group of polymers where one or both are antimicrobial, antibacterial, antiviral, a biocide, and/or antifungal.

Still referring to FIG. 2, the polymer 200 is optionally a tuned molecular weight 216. Longer chains have advantages like higher charge and/or complexing more than one object, such as a virus. Shorter chains have advantages, such as more precise charge. In general, the molecular weight of the polymer is greater than 100, 1000, 10,000, or 100,000 g/mol; less than 5,000,000, 1,000,000, or 500,000 g/mol; and/or within 5, 10, 25, or 50 percent of 100,000, 200,000, 300,000, 400,000, or 500,000 g/mol.

Still referring to FIG. 2, the polymer 200 is optionally solubility tuned 217, such as by adjusting pH of the solution, monomer size, monomer functional groups, length of the polymer 200, size of counterions, degree of salt, activity of the solution, and/or through use/application of any agent designed to complex the polymer 200 and/or attract the polymer 200 to a surface, such as the surface of a treated product, any of which may be expressed at solubility of the polymer 200 in g/L. Herein, the solution optionally and preferably contains greater then 0, 1, 2, 3, 5, 10, 15, 20, 25, 50, 75, or 95 percent water and/or any solvent such as ethanol.

Still referring to FIG. 2, the polymer 200 and/or polymer formulation is optionally viscosity tuned 218, such as in a range of centipoise units. Optionally, centipoise is expressed in units of poise, m²/s, Newton-second per square meter, and equivalents thereof. Optional ranges of viscosities of the polymer 200 and/or a formulation containing the polymer in centipoise is near water in a range of 1-5, that of blood or 15 centipoise, that of corn syrup or 50-100 centipoise, that of motor oil 150 to 500 centipoise, than of syrup or 1000-3000 centipoise, that of molasses to peanut butter or 5000 to 200,000 centipoise, or higher in a more solid state, such as lard to window putty or 1,000,000 to 100,000,000 centipoise, and/or within 10, 25, 50, 75, or 100% of any of these levels.

Still referring to FIG. 2, the polymer 200 and/or polymer formulation is optionally durability tuned 219 according to any metric, such as durability metrics in PAS2424 and/or in the more rigorous U.S. 01-1A tests, referenced supra. For instance, a durability test is to whether or not a treated substance still passes an EPA test, such as effectiveness against a microbe, bacteria, virus, and/or fungus after a series of web rub then dry cycles, such as 6 to 12 cycles, at a testing pressure, such as about 5, 10, 15, or 20 pounds of pressure plus or minus 25 or 50 percent.

Still referring to FIG. 2, the polymer 200 and/or polymer formulation is optionally surface tension tuned 220 to a range of 0.001 to 0.3 N/m; to greater than 0.01, 0.02, 0.03, 0.04 N/m; less than 0.3, 0.2, 0.1 N/m; and/or to within 10, 25, 50, 75, or 100% of 0.01, 0.02, 0.03, 0.04, 0.06, 0.08, or 1.0 N/m. For instance, a starch, slurry, paste, and/or binding agent is optionally used to increase the surface tension of the polymer 200.

Still referring to FIG. 2, in a second process, the polymer 200 is optionally tuned to form a tuned polymer counterion 230, where each of one or more counterions have antimicrobial 231, antibacterial 232, antiviral 233, and/or antifungal 234 properties. Specific properties found to have antimicrobial 231 effectiveness in association with a charged polymer are further detailed below. Generally, herein, an antimicrobial 231 is one or more of antibacterial 232, antiviral 233, and/or antifungal 234. Generally, a biocide is antimicrobial 231 and/or antibacterial 232. Still referring to FIG. 2, the first process and second process are optionally tuned at the same time; in either order, and/or are tuned in sub-parts in any simultaneous and/or time ordered manner.

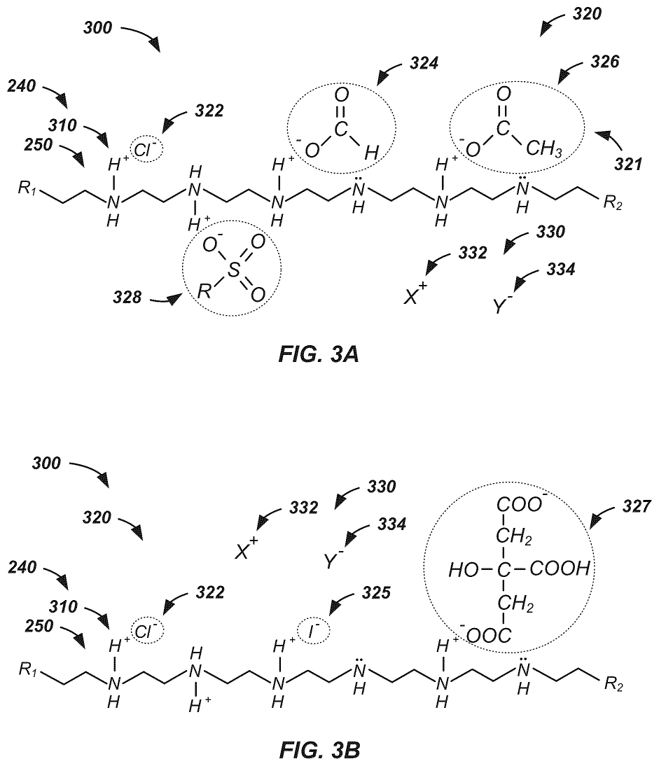

Referring now to FIG. 3A, a tuned cationic polymer 300 is described, which is an example of a polymer 200. Again, for clarity of presentation and without loss of generality, linear polyethylenimine 250 (PEI) or polyaziridine, is used as an example of a polymer 200 and/or a protonated polymer 240. Further, polyethylenimine 250 is used as a general case of linear polyethylenimine and/or branched polyethylenimine, which are further described infra.

Still referring to FIG. 3A, the polyethylenimine 250 or protonated polymer 240 is illustrated with repeating monomers, which have a carbon-carbon-nitrogen backbone, which are representative of any repeating background and/or monomer, dimer, or trimer of the polymer 200. The nitrogen is optionally protonated 310, where protonation increases with decreasing pH and decreases with increasing pH. At a pH of 4, protonation of the polyethylenimine 250 is about 30±5 percent; however, protonation is variable with treatment of the polyethylenimine 250, as further described infra. Generally, any protonation 310 level of the polymer 200 is optionally used.

Still referring to FIG. 3A, the protonation 310 sites of the protonated polymer 240 are associated with counterions 320. For instance, when polyethylenimine 250 is manufactured, polyethylenimine 250 is provided in hydrochloric acid, so the far dominant conjugate base counterion is the chloride ion 322, such as at greater than 98, 99, 99.5, or 99.9 percent. Methods of exchanging the chloride counterion 322 with another counterion are described infra. Alternatively, polyethylenimine 250 is manufactured in a “free base” form, such as a solid with a pH greater than 9, 10, 11, 12, or 13, where protonation 310 is very low, such as less than 20, 10, 5, 3, 2, 1, or 0.1 percent. Another counterion is optionally formate 324, which has at least antibacterial and/or antiviral properties, such as in combination with the protonated polymer 240 such as polyethylenimine 250. Still another counterion is optionally acetate 326, which has at least antiviral properties, such as with the protonated nitrogen and/or a protonated amine, such as in the repeating monomer of the polymer 200. Formate 324 and acetate 326 are both examples of conjugate bases of carboxylic acids 321. Yet another exemplary counterion is sulfonate 328, which is at least antimicrobial in combination with polyethylenimine 250 and/or with a cationic polymer. Generally, any anion conjugate base is optionally used as the counterion 320, such as: iodide, I⁻, bromide, Br⁻, a form of citrate or citrate²⁻, nitrate ion, NO₃⁻, sulfate ion, SO₄²⁻, nitrite, NO₂⁻, hydrogen carbonate, HCO₃⁻, hydroxide ion, OH⁻, sulfide ion, and S²⁻. Generally, any combination of 1, 2, 3, or more counterions are optionally and preferably associated with the polymer 200, such as at a charged site of the repeating monomers, such as at the protonated sites 310, and/or at functional groups that are chargeable with a reaction, such as a secondary amine in polyethylenimine 250. Further, the ratio of the 1, 2, 3, or more counterions are optionally controlled at any ratio. For instance, two different counterions are optionally controlled at a ratio of greater than 1:0, such as greater than 1:0.01, 1:0.05, 1:0.1, 1:0.2, 1:0.3, 1:0.4, or 1:0.5 and/or at a ratio of less than 0:1. Similarly three counterions are optionally held/produced at any ratio 0:1 to 0:1 to 0:1 for the relative concentrations of the three, or indeed n, counterions, where n is a positive integer greater than 1, 2, 3, 4, or 5. For instance, the ratio of three counterions is 0.1 to 0.3 to 0.6; 0.33 to 0.33 to 0.33; or any set of ratios. Further, any of the n counterions optionally and preferably are selected for one or more of the properties of being antimicrobial 231, antibacterial 232, antiviral 233, and/or antifungal 234. Referring now to FIG. 3B, the additional counterions 320 of iodide 325, I⁻, and citrate 327 in an optional basic form of citrate²⁻ are illustrated.

Still referring to FIG. 3A, the tuned polymer 210 is optionally dried into a film, is a semi-solid, and/or contains a level of salt, in solid and/or ionic form about the tuned polymer 210. As illustrated, a salt solution 330 about the tuned polymer 210 optionally contains a set of ions 332, a set of one or more cations, represented as X⁺ 332, and/or a set of one or more anions, represented as Y 334. Tuning the type and concentration of each member of the sets the cations 332 and anions 334 tunes the chemical activity coefficient, y, such as according to any version of the Debye-Hückel equation. Tuning the activity coefficient to a value greater than 0, 0.1, 0.2, 0.3, 0.4, or 0.5; to less than 1, 0.9, 0.8, 0.7, or 0.6; and/or in a range of within 10, 20, or 30% of 0.4, 0.5, 0.6, or 0.7 alters the ability of the ability of the charges on the polymer repeating units and/or their associated counterions to kill, bind with, react with, hold onto, block, and/or hinder function of the antimicrobial 231, antibacterial 232, antiviral 233, and/or antifungal 234. Thus, control of salt about the polymer 200 and/or the at least partially protonated repeating units of the polymer 200 tunes the antimicrobial 231 and/or biocide function of the tuned polymer 210.

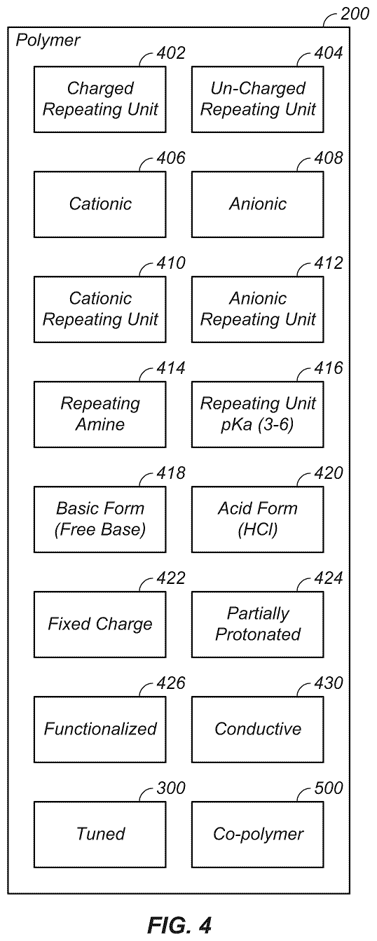

Referring now to FIG. 4, the polymer 200 is further described. Optionally and preferably, the polymer 200 is in a state comprising one or more of: a charged repeating unit 402; an uncharged repeating unit 404 and/or section; is cationic 406; is anionic 408; is a zwitterion; has a cationic repeating unit 410, has an anionic repeating unit 412; has a repeating amine 414; has a repeating chargeable monomer or polymer section; has a repeating unit with a pKa in a range of three to six 416; is in a basic form 418, such as at a pH>7; is in a PH neutral form; is in an acidic form 420, such as at a pH<7; has a fixed charge 422, such as per unit length of the polymer 200; and/or is partially protonated 424, such as on average every 1, 2, 3, 4, 5, 6, 7, 8, 9, 10, or more sets of repeating monomers.

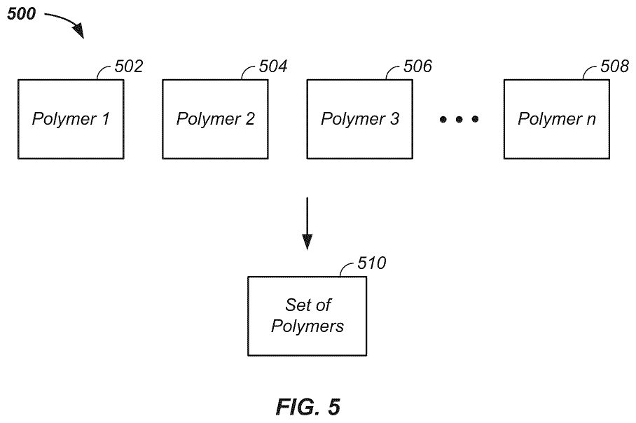

Referring now to FIG. 5, the polymer 200 is optionally a group of polymers, such as a first polymer 502, a second polymer 504, a third polymer 506, or n polymers 508, where each of the n polymers differ by any state property, such as any of the polymer states described above. Combined, the polymers 200 in a formulation comprise a set of polymers 510. For example, some have long chains than others, some have higher charges than others, some are cationic, some have a first pKa while others have a second pKa, and/or others have first counterion types and others second counterion types.

### Processes

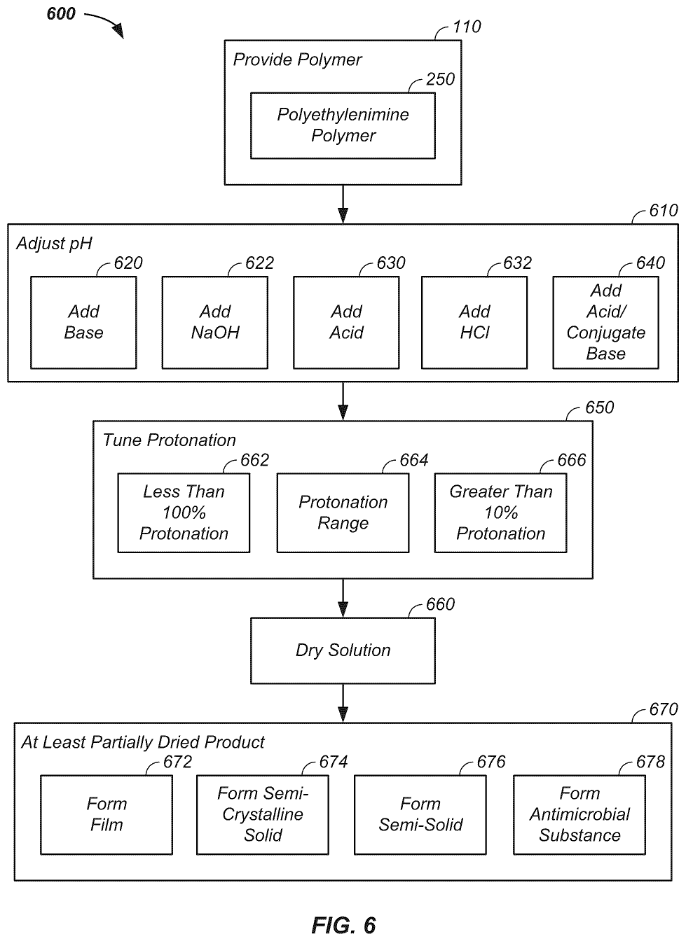

Referring now to FIGS. 6-17, exemplary process for forming the tuned polymer 210 are described. The exemplary processes are optionally combined. Further, any one or more elements/steps of one of the processes is optionally used in combination with any one or more elements/steps of another one of the processes to form the tuned polymer 210.

Referring now to FIG. 6, in a first general process 600, the tuned polymer 210 is formed by controlling/adjusting pH 610. As illustrated, in a first step the polymer is provided 110. Again, for clarity of presentation and without loss of generality, polyethylenimine 250 is used to illustrate any polymer 200. In another step, which is optionally performed in parallel with the first step, the pH is adjusted 610. For instance, the pH of the polymer 200 is adjusted, the pH of an aqueous solution containing the polymer is adjusted, and/or a pH of the polymer in any physical state, such as in a gel state, is pH adjusted. Generally, in one case a base is added 620, such as by addition of sodium hydroxide 622 in any form or concentration. Any base is optionally used in this case to reduce protonation of the polyethylenimine 250. In a second case, an acid is added 630, such as by addition of hydrochloric acid 632 in any form or concentration. Any acid is optionally used in this case to increase protonation of the polyethylenimine 250. In a third case, an acid/conjugated base 640 and/or linked pair of acid and conjugate base is used to adjust the pH 610. For instance, addition of formic acid to an acidic solution below the pKa of formic acid will increase the pH of the solution, deprotonate the polyethylenimine 250, and add a counterion of formate. Conversely, addition of formic acid to a basic solution above the pKa of formic acid will decrease the pH of the solution, protonate the polyethylenimine 250 and add a counterion of formate. Adjustment of pH to tune protonation of the polyethylenimine 250 is further described, infra. In a third step, resultant from the second step, protonation is tuned 650. For example, protonation of the polyethylenimine 250 is tuned to less than one hundred percent 662, to a protonation range 664, and/or to greater than ten percent protonation 666. Preferred protonation ranges are: greater than 0.1, 1, 5, 10, 15, 20, or 30 percent; less than 100, 99, 90, 80, 70, 60, or 50 percent; and/or in a range of ±0, 1, 5, 10, 15, 20, 25, or 50 percent of any of 30, 35, 40, 45, 50, 55, 60, 65, or 70 percent, where any combination of ranges described herein are optionally used, such as 35-45 percent and/or 30-40±5 percent on either end. A protonation range of 20-60 percent is the preferred state of the tuned protonation 650.

Still referring to FIG. 6, before, during, and/or after the steps of providing the polymer 110, adjusting pH 610, and/or tuning protonation 650, a step of drying 660 is optionally used. The drying step 650 forms an at least partially dried substance/product 670, such as a where a film is formed 672, a form or semi-crystalline solid is formed 674, a semi-solid 676/gel is formed, a wet film, a dry film, and/or an antimicrobial substance 678 is formed/concentrated. Generally, the drying step 660 increases the concentration of the polyethylenimine 250, the counterions 320, and/or the salt solution 330.

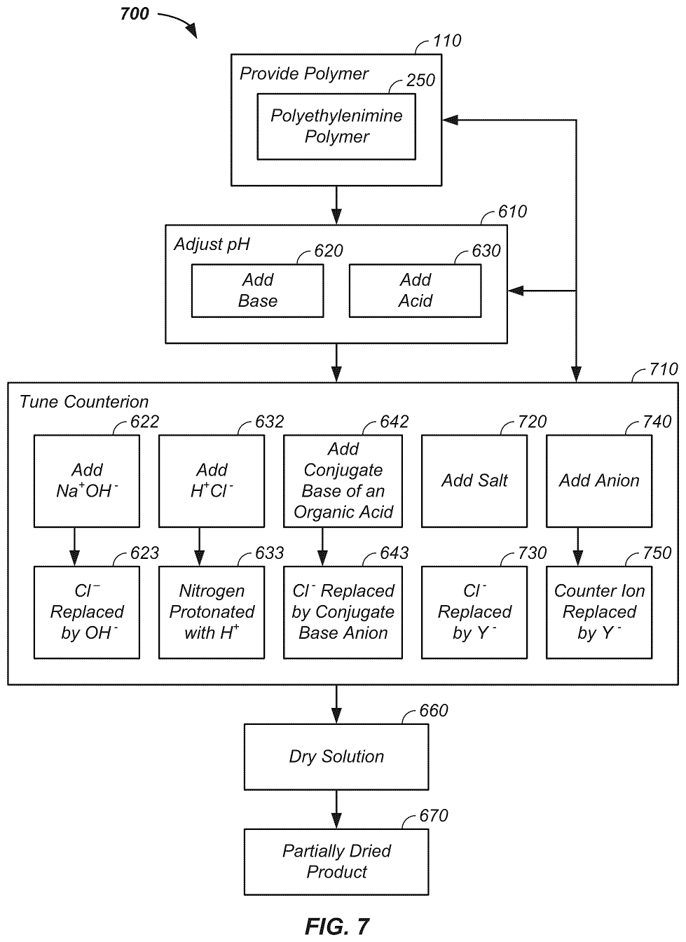

Referring now to FIG. 7, in a second general process, the tuned polymer 210 is formed by controlling and/or adjusting pH 610 to yield a tuned counterion solution 700/formulation. Generally, the steps of tuning the protonation 650, illustrated in FIG. 6, are optionally and preferably performed in combination with the steps of tuning the counterion solution 710. A process of tuning the counterion 710 optionally includes any process of controlling one or more counterion types, ions, and/or molecules associated with cations or charges on the repeating units of the polymer 200. Exemplary methods of tuning the counterion 710 comprise: (1) adding sodium hydroxide 622, in any concentration, for replacing chloride counterions with hydroxide counterions 623 on the polyethylenimine 250; (2) adding hydrochloric acid 632, in any concentration, to protonate the polyethylenimine 250, a secondary amine, and/or any protonatable functional group of the polymer 200; (3) adding a conjugate base of an organic acid 642 to replace the chloride counterion with the conjugate base anion 643, where more generally the conjugate base anion optionally displaces any counter anion associated with the polyethylenimine 250; (4) adding salt, such as where the chloride ion, the anion 334, Y, or indeed any counter anion is replaced with one or more added salt anions; (5) adding an anion 740 where the counterion associated with polyethylenimine 250 is replaced by the anion 334, Y, and/or (6) using any like substitution methods of anions, such as via an anion exchange column/method. Again, the resulting solution/compound/formulation is optionally dried 660 to form at least a partially dried product 670, as described supra. For clarity, in an example of adding sodium formate and/or formic acid, the added formate anion is used to replace a counterion associated with polyethylenimine 250, such as the chloride ion, such as when starting with a protonated form of polyethylenimine 250 in hydrochloric acid, as further described infra. Similarly, addition of sodium acetate and/or acetic acid adds the counterion of acetate to the polyethylenimine 250 and displaces counterions associated with polyethylenimine 250, where the overall process of tuning the counterion solution 700 tunes/controls the mean percentage of any 1, 2, 3, or more counterions associated with the polyethylenimine 250.

Still referring to FIG. 7 and referring again to FIG. 3, a carboxylic acid and/or a conjugate base of a carboxylic acid 321 is optionally used to displace the chloride ion, such as in step three of the previous paragraph, and/or more generally to supply a counterion. For instance, formic acid, a carboxylic acid, is used to provide formate (a conjugate base of formic acid) and/or a proton to any formulation herein. Similarly, acetic acid, also a carboxylic acid, is used to provide acetate (a conjugate base of acetic acid) and/or a proton to any formulation herein. Additional carboxylic acids that are optionally used to supply counterions and/or other counterions isolated from carboxylic acid include any of: acetic acid (ethanoic acid); formic acid (methanoic acid); citric acid; propionic acid (propanoic acid); butyric acid (butanoic acid); benzoic acid; lactic acid; tartaric acid; malic acid; oxalic acid; succinic acid; palmitic acid; stearic acid; oleic acid; and/or linoleic acid.

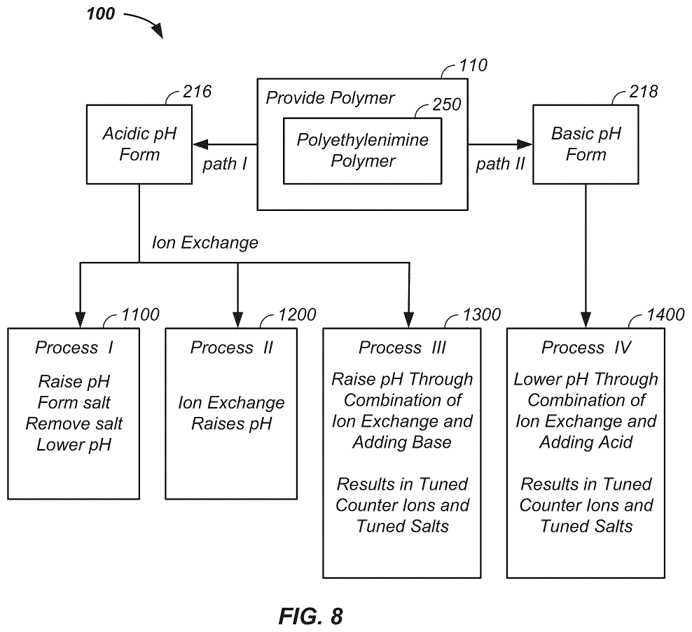

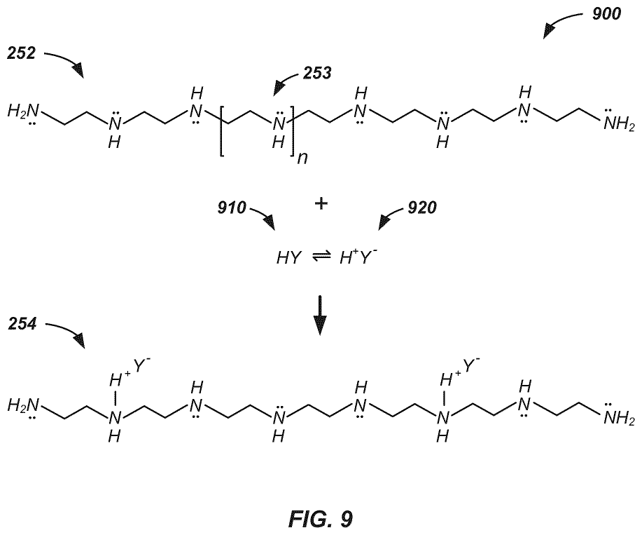

Referring now to FIGS. 8-11, for clarity of presentation and without loss of generality, four exemplary processes of tuning protonation of the polymer 650, tuning counterions 710 of the polymer 200, and/or tuning the salt solution 330 about the polymer 200 are described. Referring now to FIG. 8, a flowchart of the four exemplary process is provided, where an initial polymer 110, such as polyethylenimine 250, is pH adjusted 610 along a first path to form an acidic pH form 216 of the polyethylenimine 250 and/or along a second path to form a basic pH form 218 of the polyethylenimine 250. Three processes using the acidic pH form 216 are illustrated: (1) a first process of alkalization polymer tuning 1100 that raises pH of the polyethylenimine 250, forms a salt, removes the formed salt, and lowers the pH to tune the polyethylenimine 250; (2) a second process of alkalization polymer tuning 1200 that uses ion exchange to raise the pH about the polyethylenimine 250 to tune the polyethylenimine 250; and/or (3) a third process of alkalization polymer tuning 1300 that raises the pH about the polyethylenimine 250 through linear and/or sequential addition of a base and ion exchange, in either order of addition, to tune the polyethylenimine 250 and associated counterions. One process using the basic form 218 is illustrated: (4) a fourth acidification polymer tuning process 1400 of lowering the pH of the polyethylenimine 250 through a combination of adding acid and/or use of ion exchange to yield a tuned polyethylenimine 250 in terms of protonation, counterions, and/or salt solution. The fourth process 1400 optionally has sub-parts associated with each of processes I-III, albeit in reverse direction of pH adjustment. Each of the four processes are further described in the following examples. However, first generic processes of tuning with a conjugate base are illustrated in two subprocesses. Referring now to FIG. 9, a first subprocess of using a conjugate base to acidify 900 the polymer 200, such as with repeating monomers 253, such as polyethylenimine 250, is illustrated. As illustrated, a basic form of polyethylenimine 252 is interacted (reacted and/or supplied) with an acid 910—conjugate base 920 pair or indeed with just the acid 910 or with just the conjugate base 920, where an equilibrium between the acid 910 and the conjugate base 920 suffices to continue to provide each of the acid 910 and/or the conjugate base in the reaction/counterion supply. Generally, the acid or H⁺ protonates the polyethylenimine 250 to form a tuned protonation level of the polyethylenimine 254 and the salt anions, Y, provide the counterions 320. Notably, when a weak acid, such as formate, acetate, citrate, any monoprotic acid, and/or any dibasic acid, is used to acidify the polyethylenimine 250 to form a tuned protonation level of the polyethylenimine 254, little to no salt is formed. In the strict case of a pure weak acid with no impurities, no salt is formed. However, salt concentrations are typically held to less than 20, 10, 5, 4, 3, 2, 1, 0.5 or 0.1 percent with this approach, such as due to presence of impurities in the reagents/solvent(s), such as the presence of sodium carbonate in the water.

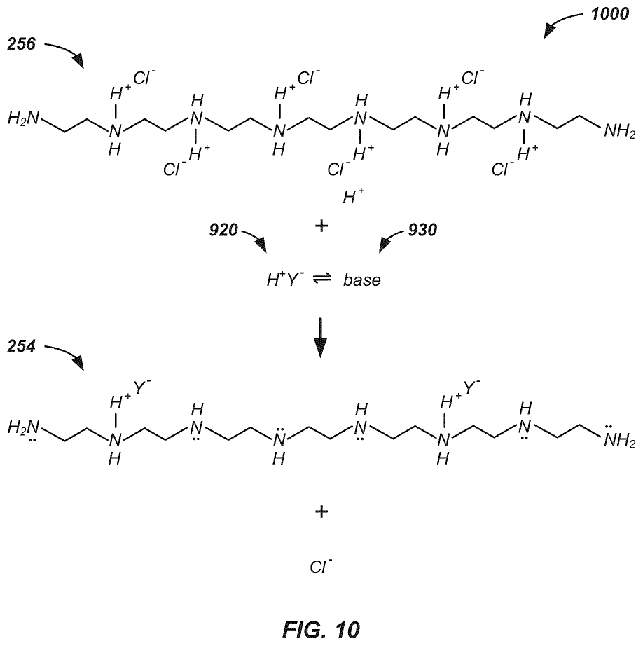

Referring now to FIG. 10, a second subprocess of using a conjugate base to alkalize 1000 the polymer 200, such as polyethylenimine 250, is illustrated. As illustrated, an acidic form of polyethylenimine 256 is interacted (reacted and/or supplied) with an acid 910—conjugate base 920 pair or indeed with just the acid 910 and/or with just the conjugate base 920, where an equilibrium between the acid 910 and the conjugate base 920 suffices to continue to provide each of the acid 910 and the conjugate base in the reaction/counterion supply. Generally, the conjugate base 920 deprotonates the acidic form of the polyethylenimine 256 to form a tuned protonation level of the polyethylenimine 254 and the salt anions, Y, provide the counterions 320. Notably, when a weak acid, such as formate, acetate, or citrate, is used alkalize the acidic form of the polyethylenimine 256 to form a tuned protonation level of the polyethylenimine 254, salt is formed. For example, when the acidic form of the polyethylenimine 256, such as formed with hydrochloric acid, has the counterion of chloride 322, is used/supplied, the original counterion (Cl⁻) associated with the acidic form of the polyethylenimine 256 is displaced to form part of the salt solution 330. Thus, the first process has an advantage of limiting to eliminating production of the salt solution 330 about the tuned protonation level of the polyethylenimine 254 and/or the second process has an advantage of yielding salt and optionally a controlled amount of salt in the tuned protonation level of the polyethylenimine 254, which affects the antimicrobial 231 properties of the composition. Yet, it is noted that the resulting tuned cationic polymer 300 differs using the first process compared to using the second process without use of an additional optional salt control step, such as further described infra.

### Example I

### Process I

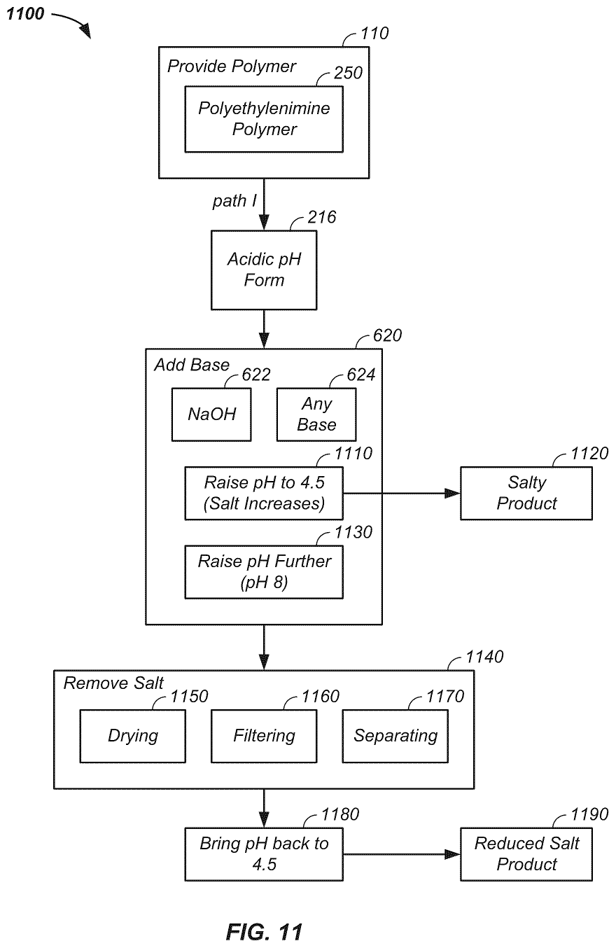

Referring now to FIG. 11, the first alkalization polymer tuning process 1100 is further described. As described, supra, generally the first alkalization polymer tuning process 1100 provides a polymer 110, such as polyethylenimine 250, such as in an acidic pH form 216; adds a base 620 to raise the pH, which yields a salt; in a subsequent and/or concurrent step removes at least a portion of the formed salt 1140; and adds an acid to bring the pH back down. Generally, the process of raising the pH, such as with sodium hydroxide 622, in any form or concentration, and/or adding any base 624, such as even a conjugate base, raises the pH of the formulation containing polyethylenimine 250 to a level where salt is formed, such as to a raised pH level of 4.5 1110, which yields a salty product 1120 containing polyethylenimine 250 or raises the pH even further 1130, such as to a pH greater than 4, 5, 6, 7, 8, or 9, which generates even more salt. Essentially, forming the salt, which precipitates, allows for an easy removal of the salt, whether in a precipitate form or in solution, through the process of removing the salt 1140.

Generally, the salt removal step 1140 optionally includes one or more of the steps of drying 1150, filtering 1160, and/or separating 1170 the salt with any salt removal technique, such as centrifugation, washing, rinsing, decanting, and the like. Once the salt is removed, the polyethylenimine 250 is brought back down in pH 1180, such as to a target pH of less than 6, 5.5, 5, 4.5, 4, 3.5, or 3 and/or to greater than 2, 3, or 4, which reprotonates the polyethylenimine 250—yielding the antimicrobial 231, antibacterial 232, antiviral 233, and/or antifungal 234 properties, described supra, associated with the more protonated form of polyethylenimine 250 at the lower pH and with a reduced salt concentration 1190/chemical activity allowing better interaction with bacteria and especially viruses, in any form.

### Example II

### Process II

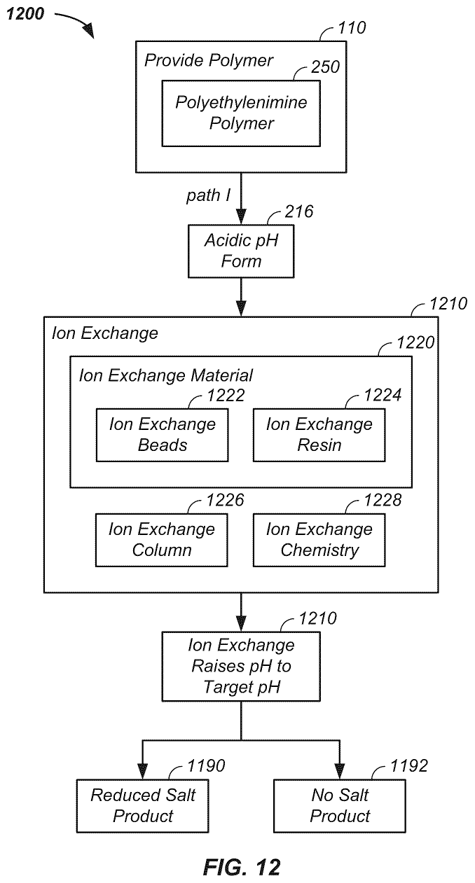

Referring now to FIG. 12, the second alkalization polymer tuning process 1200 is further described. In the second alkalization polymer tuning process 1200, the polymer is provided 110, such as the polyethylenimine 250, in a solution, such as in acidic pH form 216 and ion exchange 1210 is used to increase the pH of the polyethylenimine 250 formulation. For example, an ion exchange material 1220, such as ion exchange beads 1222 and/or an in exchange resin 1224 is used in an ion exchange column 1226, a container, and/or in ion exchange chemistry 1228 to exchange ions/counterions in/on the polyethylenimine 250 for anions on the anion exchange material. Optionally and preferably, anions on the ion exchange material 1220 are exchanged for counterions associated with positively charged sites on the polymer 200, such as for chloride anions functioning as counterions to protonated secondary amines of the polyethylenimine 250. Optionally and preferably, greater than 1, 2, 5, 10, 15, 20, 25, 30, 40, 50, 60, 70, 80, 90, 95, or 99 percent of counterions/counter-anions, such as chloride anions functioning as counterions to the polymer 200 are adsorbed onto and/or are bound to the ion exchange material 1220. Optionally and preferably, the percentage of counterions to protonated sites on the polymer, such as the percentage of chloride ions, is reduced to less than 99, 95, 90, 80, 70, 60, 50, 40, 30, 20, 10, or 5 percent of a total number of counterions, such as at a fixed point in time. The ion exchange material 1220 is optionally used to provide any anion to the polymer solution, such formate, acetate, iodide, and/or any anion described herein. In one optional and preferably case, the ion exchange material 1220 exchanges hydroxide anion, OH⁻, for the chloride anions. The hydroxide anions and protons of the acidic solution combine to form water, which raises the pH of the solution, such as to a pH greater than 2, 3, 4, 5, or any pH described herein. Essentially, the ion exchange process 1210 achieves a task of raising the pH of the polyethylenimine 250 to a tuned pH level. During the process of raising the pH, the salt concentration of the polymer and/or a concentration of anions/counterions in the solution containing the polymer 200 is reduced, is not increased, and/or is raised by less than 10, 5, 2, 1, or 0.1 percent. Optionally, the ion exchange process 1210 yields a target pH based on pKas and/or binding/exchange coefficients of the exchange material. For instance, a formate exchange naturally bring the pH to within a pH unit or two of a pKa of formate of 3.75 or for acetate of 4.75. Combinations of conjugate bases are optionally used in the ion exchange process, as further described infra. Notably, commercially available polyethylenimine 250 is in either a fully protonated form with a pH of about 1 with greater than 90, 95, or 99% of the counterions to the protonated amines being chloride anions or is available in a fully deprotonated state with no charges amines at a pH greater than about 12. Commercially available polyethylenimine with a pH of less than six is not available in any form except where greater than 99% of the counterions, to protonated amines of the polyethylenimine, are chloride.

### Example III

### Process III

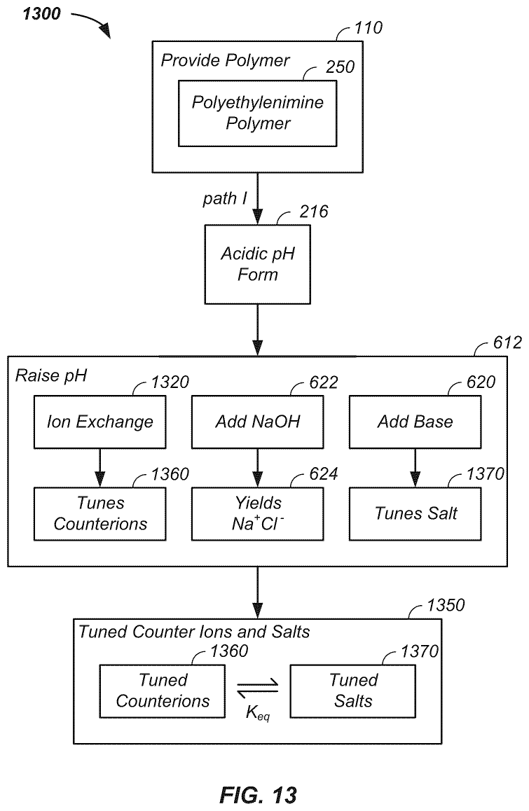

Referring now to FIG. 13, the third alkalization polymer tuning process 1300 is further described. Generally, the third alkalization polymer tuning process 1300 combines elements of the first alkalization polymer tuning process 1100 and the second alkalization polymer tuning process 1200 to use both ion exchange 1320 and addition of a base 620 to raise the pH 612 to a tuned pH/protonation level of the polyethylenimine 250. More specifically, the third alkalization polymer tuning process 1300 provides a polymer 110, such as the polyethylenimine 250 in an acidic form 216 and raises the pH 612 of the formulation, such as through one and preferably two or more of: (1) ion exchange 1320, which tunes the counter ions 1360; (2) addition of sodium hydroxide 622, which yields sodium hydroxide ions 624; and/or (3) adding a base 620, which tunes the salt 1370/counterions. For example, if formic acid/formate is added to raise the pH 612, the formate anion 324 is generated in the aqueous salt solution in the formulation about the polyethylenimine 250 and/or the formate anion 324 is provided as the counterion to the polyethylenimine 250, such as replacing, with an equilibrium coefficient, the chloride ion 322. Similarly, any acid/conjugate base molecule or acid/base pair is optionally used in place of and/or in combination with the formic acid:formate acid/base pair, which allows tuning of at least one of: (1) the salt type and/or salt concentration in the formulation about the polyethylenimine 250 and/or (2) the type and/or concentration of the counterions associated with the protonated sites 310 of the polyethylenimine 250.

### Example IV

### Process IV

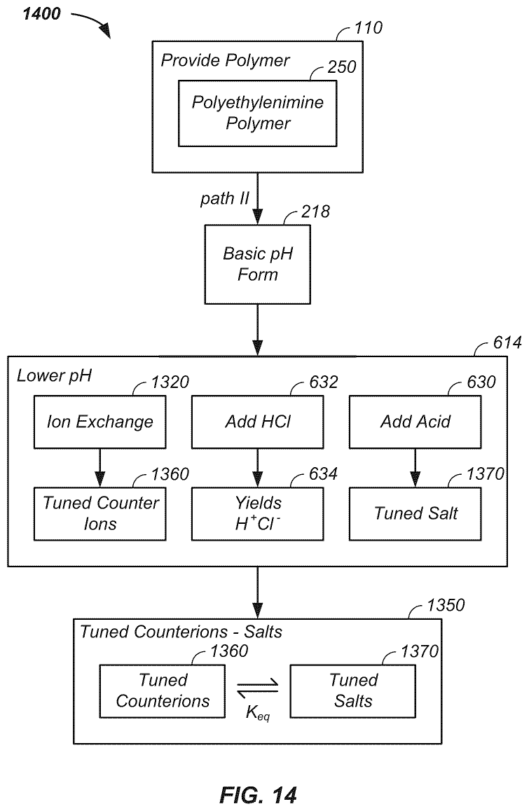

Referring now to FIG. 14, the acidification polymer tuning process 1400 is further described. The acidification polymer tuning process 1400 is essentially any combination of the first alkalization polymer tuning process 1100, the second alkalization polymer tuning process 1200, and the third alkalization polymer tuning process 1300, where the pH is lowered rather than raised. For clarity of presentation and without loss of generality, only one of the inverse pH adjustment processes is here illustrated in FIG. 14. Generally, the acidification polymer tuning process 1400 provides a polymer 110, such as polyethylenimine 250, in the basic pH form 218; the pH is lowered 614; and the counterions and/or salts in solution are tuned 1350 in serial and/or parallel steps. In the lowering pH step 614, one and preferably two or more pH lowering processes are used, such as: (1) using ion exchange 1320 to tune the counterions 1360; (2) adding hydrochloric acid 632 to yield H⁺ and Cl⁻ in the formulation; and/or (3) adding acid 630 to tune the salt 1370 formed in solution. Again, in a manner related to that described in the third alkalization polymer tuning process 1300, addition of an acid-conjugate base pair, such as a formic acid-formate pair, tunes the counterion 320, such as associated with the protonated sites 310 of the polyethylenimine 250. Further, the pKa of the acid-conjugate base pair buffers the formulation. For instance, addition of the formic acid-formate acid/base pair results in lowering of the pH 614 while providing a buffer capacity, related to the amount of the acid/base pair added, about the pKa of the formic acid-formate pka of 3.75. Similarly, addition of the acetic acid-acetate acid/base pair buffers the pH of the formulation of the polyethylenimine 250 about the pKa of 4.75 for the acetic acid-acetate acid/base pair. Notably, the tuned counterions-salts 1350 are an equilibrium of tuned counterions 1360 with tuned salts 1370, as further described infra. Generally, the acidification polymer tuning process 1400: (1) lowers the pH with ion exchange and/or addition of an acid; (2) optionally provides the proton for the protonation 310 via the use of a weak acid-conjugate base acid/base pair, such as the use of formic acid; (3) tunes availability of the type and/or concentration of the counterion 320 via choice of the weak acid/base pairs or choice of strong acid type; (4) is optionally used in a process of creating no or limited salt, such as described in the process of using a conjugate base to acidify 900 the polymer 200, as described supra; (5) provides a buffer related to the pKas of the added acids; and/or (6) optionally and preferably removes a filtering step associated with removing excess salt in the formulation, such as resulting from either use of the initial fully acidic form of polyethylenimine 256 and/or the second subprocess of using a conjugate base to alkalize 1000 the polymer 200, which yields a salt, as described supra.

Referring again to FIG. 14 and generally referring to the salt solution 330 and the counterions 320 associated with the polymer 200, the salts in the salt solution 330 are in equilibrium with the counterions 320 associated with the polymer 200 in terms of position. That is, a first counterion and a first salt in solution optionally swap positions, such as relative to a protonation site 310 of the cationic 211 polymer. However, the first counterion type and the second counterion type have preferred locations. For instance, chemically the formate 324 will tend to displace the chloride ion 322 as the counterion. Further, since the polyethylenimine 250 preferably has thousands of protonated sites 310, the probability is great that the counterion type with the greater attraction to the protonated site 310 will be associated with the polymer 200. This probability of knowing which anion type is the counterion 320 and which anion type is in the salt solution 330 only increases with chain length of the polymer 200, such as the polyethylenimine 250. Generally, one can quantify the counterion 320 to salt solution 330 mean likelihood as being greater than 1, 2, 5, 10, 20, 25, 50, 75, 80, or 90 percent a counterion or vise-versa, such as by applying the relevant position equilibrium.

### Example V

### Process V

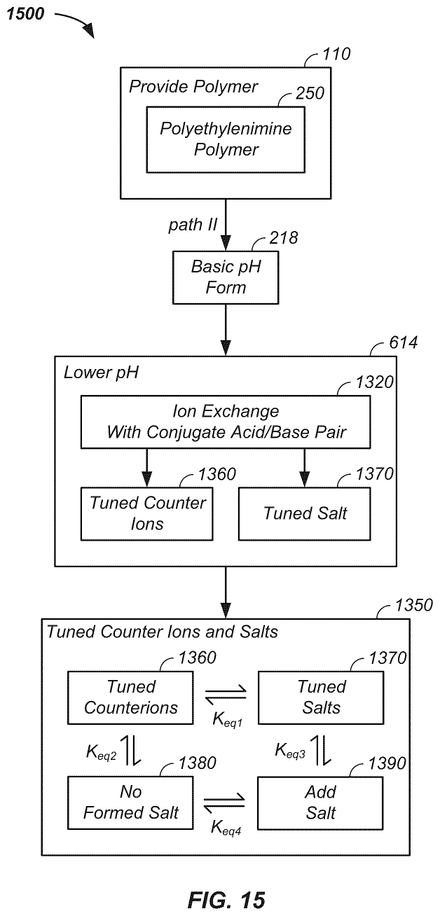

Referring now to FIG. 15 and referring again to FIG. 9, a conjugate base polymer tuning process 1500 is illustrated. Generally, two or more stock solutions are optionally mixed to form the tuned polymer 210, which is particularly beneficial when starting with the basic form of polyethylenimine 252 or any polymer 200. Generally, a polymer is provided 110, such as polyethylenimine 250 in the basic pH form 218 and the pH is lowered 614, such as with an ion exchange conjugate acid/base pair 1320, which tunes the counterions 1360 and tunes the salt 1370. An example is provided for clarity of presentation and without loss of generality. If the basic form of polyethylenimine 252, such as in a first stock solution, is mixed with an acid/base pair or simply an acid, such as formic acid, in a second stock solution the basic form of polyethylenimine 252 is provided with an proton, from the acid, to yield a tuned protonation level of the polyethylenimine 254 and the counterion 320 is provided as the conjugate base. In a more specific example, if the basic form of polyethylenimine 252, such as in a first stock solution, is mixed with a second stock solution of formic acid, the formic acid concentration quantitatively yields an amount of the protonation sites 310 and yields the counterion 320 of formate 324 at a quantitative level, such as according to the interaction illustrated in the first subprocess of using a conjugate base to acidify 900 the polymer 200 illustrated in FIG. 9. Notably, this allows for direct quantitative measurement and/or comparison of the efficacy of differing counterions 320 associated with the polymer 200. For instance, a first formulation is made with a controlled amount of protonation 310 and a controlled amount of the counterion formate 324, which is tested against a bacteria or virus, such as using a government regulated process, as described supra. Similarly, a second formulation is made with a controlled amount of protonation 310 and a controlled amount of the counterion acetate 326, which is tested against a bacteria or virus, such as using a government regulated process, as described supra. Hence, each protonation level 310 and each controlled amount of counterion 320 is thus optionally and preferably controlled and quantitatively tested, as further described infra. Further, combinations of counterions 320 are optionally tested. For instance, in a first test a controlled amount of formate is added and tested yielding a first efficacy measure and in a second test, 100 ppm iodide anion is added, tested, and a second efficacy is measured, yielding, by difference, the effectiveness of the iodide anion. Further, the conjugate base polymer tuning process 1500 described herein logically combines with and/or is used as a step in any of the first general process 600, the second general process yielding a tuned counterion solution 700/formulation, the first alkalization polymer tuning process 1100, the second alkalization polymer tuning process 1200, the third alkalization polymer tuning process 1300, and/or the acidification polymer tuning process 1400, such as where a change in pH step is used, a counterion 320 is introduced, and/or a salt solution 330 is controlled.

Still referring to FIG. 15, the tuned counterions 1360, the tuned salts 1370, no formed salt state 1380, and/or the added salt state 1390 are all in equilibrium with each other. However, the chemical properties of the ions, the activity of the solution, relative concentrations, and/or the probabilities associated with a large number of protonated sites 310 allows for calculation and/or probability determinations of which ions, such as the counterions 320, are associated with the polyethylenimine 250, as described supra.

### Example VI

### Preprocessing

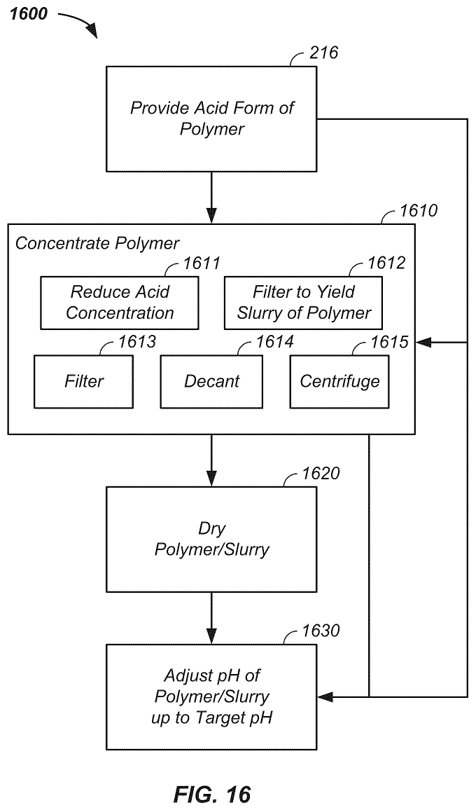

Referring now to FIG. 16, a polymer slurry acid reduction process 1600 is illustrated. Essential, the polymer slurry acid reduction process 1600 is optionally used in conjunction with any of the above described methods; however, a preferred use of the polymer slurry acid reduction process 1600 is to concentration the polymer 200 and/or reduce the overall acid molarity, which results in less salt production, where too much salt may interfere with efficacy of the tuned polymer 210 in terms of being antimicrobial 231, antibacterial 232, antiviral 233, and/or antifungal 234. Generally, an acidic form of the polymer is provided 216, such as the acidic form of polyethylenimine 256. The acidic form of polyethylenimine 256 is concentrated or simply the polymer is concentrated 1610, such as by one or more of: reducing the acid concentration 1611, filtering the acidic form of polyethylenimine 256 in hydrochloric acid to yield a slurry of polymer 1612, filtering 1613, decanting 1614, centrifuging 1615, or any process that reduces the overall molarity of the hydrochloric acid in any intermediate or final formulation of the tuned polymer 210. Essentially, removal of a portion of the hydrochloric acid results in a reduction of the amount of the chloride ion 322 and hence the reduction of the salt sodium chloride when the above described processes are implemented, where some of the processes exchange the chloride counterion 322 for another ion and/or alter the pH to form sodium chloride salt, such as by addition of a base, such as sodium hydroxide where the sodium cation combines with the chloride anion to form sodium chloride in any physical state, such as in solution and/or as a precipitate. As illustrated, the resulting polymer/slurry is optionally dried 1620, as described supra, and/or is pH adjusted up to a target pH 1630, as described supra. Stated again, the filtering step 1613 and/or the step of concentrating the polymer 1610 reduces the total base molarity needed adjust the pH, so less salt is generated with the use of the polymer slurry acid reduction process 1600, especially when raising the pH of the formulation from a very acidic pH, such as less than 3, 2, 1.7, 1.6, 1.5, or 1.

### Example VI

### Preprocessing

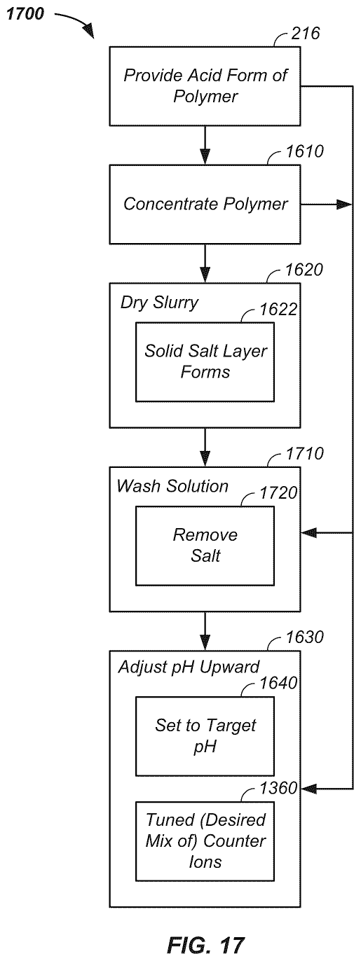

Referring now to FIG. 17, a polymer-salt pair salt reduction process 1700 is illustrated. Generally, the polymer-salt pair salt reduction process 1700 optionally uses any one of more steps of the polymer slurry acid reduction process 1600, described infra, such as the step of concentrating the polymer 1610 and/or drying the slurry 1620, which may result in formation of a solid salt layer 1622. With or without formation of the salt layer 1622, the initial acidic form of the polymer 216, such as the acidic form of polyethylenimine 256, is optionally washed 1710, such as with an aqueous solution, to remove/reduce salt 1720. Again, optionally the pH is adjusted upward 1630 at the same or later time to set the pH to a target pH 1640 and/or to yield a tuned (desired mix/type/concentration) of counter ions 1360, as described throughout. Again, the polymer-salt pair salt reduction process 1700 is used with any method described herein to remove formed salt.

### Polymer Tuning

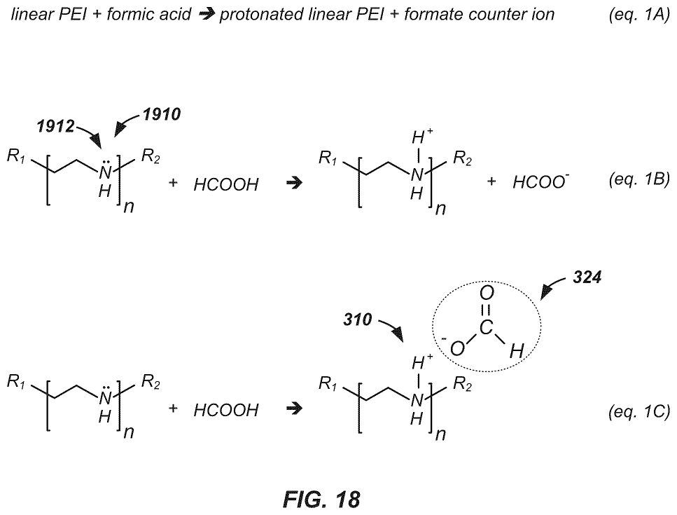

Referring now to FIGS. 18-28, details on counterion tuning are provided for clarity of presentation and without loss of generality by way of the examples provided, infra.

### Example I

Referring now to FIG. 18, a polymer tuning with a weak acid process 1800 is illustrated. In this example, any strong or weak acid is optionally substituted for the illustrated process of adding the counter ion formate 324 to the polymer 200 or as illustrated to the polyethylenimine 250. Referring now to equation 1, in FIG. 18, the reaction of:

$$linear PEI + formic acid \to protonated linear PEI + formate ion \qquad ( eq . 1 )$$

is repeated with chemical illustrations in equations 1B and equation 1C. Generally, the formate counter ion 324 from the formic acid equilibrium with formate is added to a proton on the secondary amine 1912 of the repeating unit 1910 and/or a proton from the formic acid protonates 310 the secondary amine 1912 to form a cationic polymer repeating unit.

### Example I

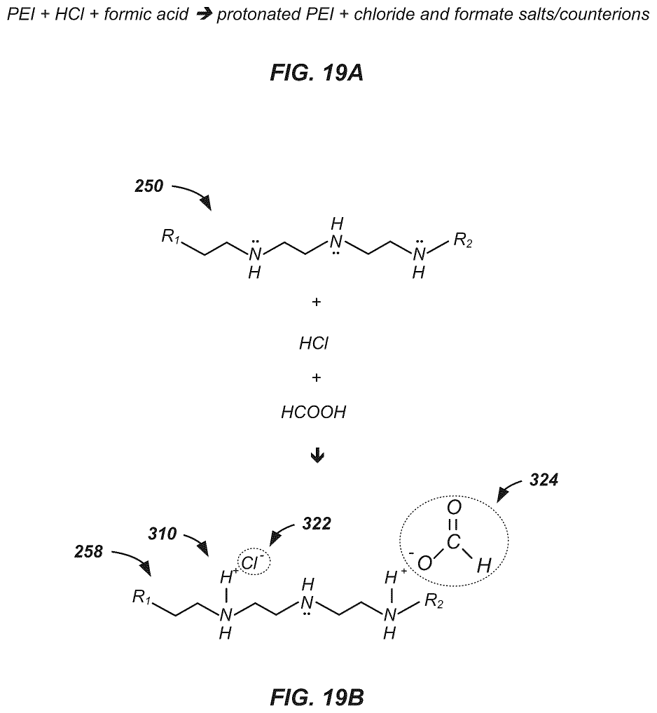

Referring now to FIG. 19, a polymer tuning with a weak acid and salt production process 1900 is illustrated. Generally, the weak acid and salt production process 1900 is the polymer tuning with a weak acid process 1800 with the addition of another acid, such as a strong acid, such as hydrochloric acid at any concentration or state. Again in this example, any strong or weak acid is optionally substituted for either of the illustrated processes of adding hydrochloric acid and the counter ion formate 324 to the polymer 200 or as illustrated to the polyethylenimine 250. Referring now to equation 2, in FIG. 19, the reaction of:

$$PEI + HCl + formic acid \to protonated PEI + chloride ion + formate ion \qquad ( eq . 2 )$$

yields: (1) two protonation sites 310 on the protonated PEI having a non-chloride counterion, one proton on average from the hydrochloric acid and one from the formic acid and (2) two counterions 320, a chloride anion 322 and a formate anion 324 from the two acids, respectively. Generally, the chloride ion 322 and the formate counter ion 324 from the formic acid-formate equilibrium is added as being loosely bound to the protons on the secondary amines 1912 of the repeating unit 1910 and/or protons from the polyethylenimine 250 and/or formic acid protonates 310 the secondary amine 1912 to form a cationic polymer repeating unit. Herein, the result of a protonated polymer having non-chloride counterions 258 is distinct from a protonated polymer having only chloride counterions, both in chemical structure and application efficacy.

### Tuning Counterion Availability

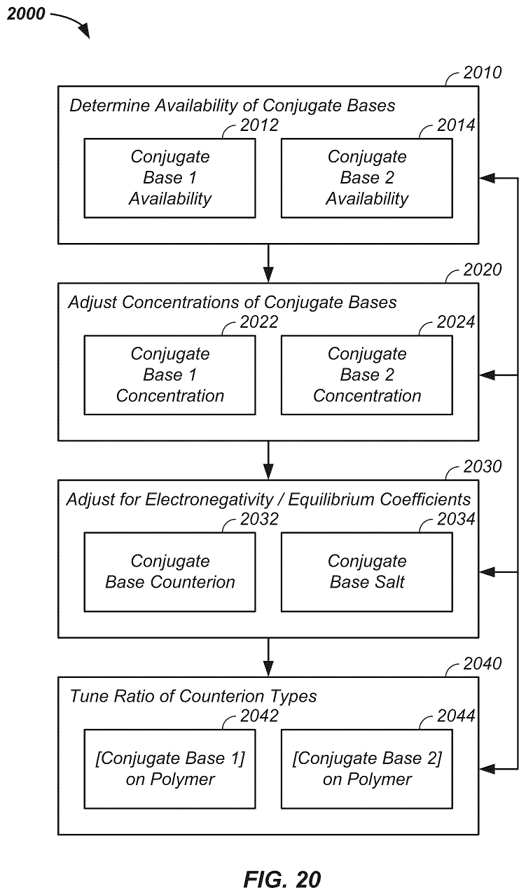

Referring now to FIG. 20 and FIGS. 21-28, relative concentration of conjugate base tuning 2000 is illustrated. Generally, a process of relative concentration of conjugate base tuning 2000 includes one or more of the steps of: (1) determining availability of the conjugate bases 2010/anions/counterions, such as the availability of a first conjugate base 2012, a second conjugate base 2014, or n conjugate bases, where n is a positive integer greater than 0, 1, 2, 3, 4, or 5; (2) adjusting concentration of the conjugate bases, such as a first conjugate base concentration 2022, a second conjugate base concentration 2024, or n conjugate base concentrations, where n is a positive integer greater than 0, 1, 2, 3, 4, or 5; and/or (3) optionally adjusting for electronegativity and/or equilibrium coefficients 2030, such as determining/using an equilibrium between a conjugate base associated with the polymer 2032 and the conjugate base being a salt in solution 2034 and/or the affect of electronegativity on an activity coefficient for the protonated site 310/counterion 320, which combined and/or separately allow for tuning a ratio of counterion types 2040, such as a concentration of first conjugate base on the polymer association 2042, a concentration of a second conjugate base on the polymer association 2044; or concentrations of n conjugate bases on the polymer associations, where n is a positive integer greater than 0, 1, 2, 3, 4, or 5. For clarity of presentation and without loss of generality, several examples are provided, infra, to further illustrate the process of relative concentration of conjugate base tuning 2000.

### Example I

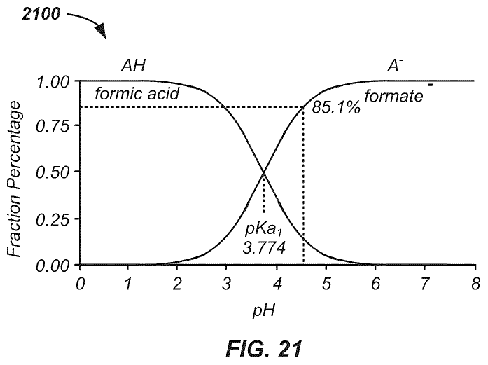

Referring now to FIG. 21, a process of determining a first conjugate base availability 2100 is illustrated. Generally, the amount of any counterion 320 associated with the polymer 200 in a formulation of the tuned polymer 210 is optionally and preferably controlled. For clarity of presentation and without loss of generality, control of the counterion formate 324 is illustrated, which again is optionally repeated for any counterion and/or two or more counterions 320. Optionally, the counterion concentrations and/or availabilities are optionally independently quantitatively controlled, such as through implementation of any one or more of the processes described herein. In the case of formate 324, formate has a pKa of 3.774. Based upon the pKa, the fraction percentage of any form or any monoprotic, diprotic, and/or triprotic acid is calculable. In FIG. 21, the fraction percentage of formic acid and formate are illustrated as a function of pH. As indicated, if a fraction percentage of formate of 85.1 percent is desired, then the pH is adjusted to pH 4.5. Conversely, the pH is selected and the fraction percent of the conjugate base(s) are known. Hence, using any of the processes described herein, the formate concentration is 85.1 percent of the total molarity of the formic acid-formate acid/base pair at pH 4.5. Notably, the overall formate and/or conjugate base concentration is optionally raised or lowered to alter the total amount/concentration of formate 324/conjugate base available as a counterion 320 in a formulation of the tuned polymer 210, as further described in the following example.

### Example II

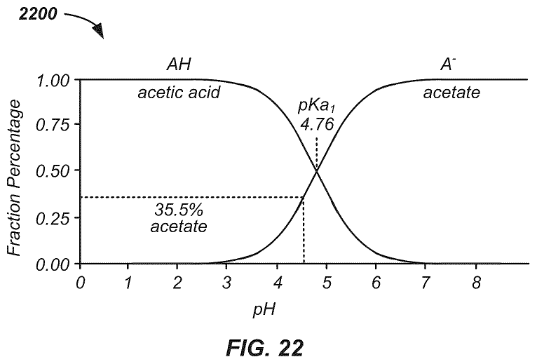

Referring now to FIG. 22, second conjugate base availability determination 2200 is illustrated. In this example, availability of acetate 326 is determined just as described for the determination of formate 324 in the prior example, only using a pKa of acetate of 4.76. While formate 324 had an availability of 85.1 percent at pH 4.5, as described in the preceding paragraph, acetate 326 only has an availability of 35.5 percent at the same pH of 4.5. Thus, if both formate 324 and acetate 326 were added in equal molar values, the ratio of formate-to-acetate is 85.1:35.5 at pH 4.5, or a ratio of 2.4:1. However, the total mass/concentration of each counterion is optionally varied/controlled, as further described in the following example.

### Example III

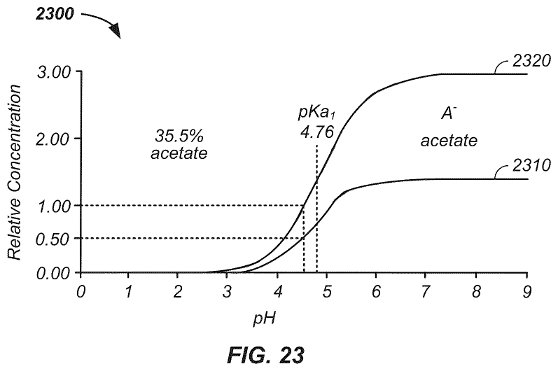

Referring now to FIG. 23, adjusting a conjugate base concentration 2300 is illustrated. Generally, any counterion 320 concentration is raised/lowered by altering the total mass in a formulation of the polymer 200. In this illustrative example, acetate 326 is illustrated at a first acetate concentration 2310 and at a second acetate concentration 2320 that is two times the first concentration. Thus, by increasing the acetate concentration, the ratio of formate-to-acetate is controlled, as illustrated in the next example. Stated another way, while the fraction of acetate to total acetate+acetic acid is still 35.5 percent at pH 4.5, as illustrated in the prior example, the amount of acetate available is controllably altered by changing the total mass of acetate+acetic acid in the formulation and/or in a formulation step. For instance, doubling or halving the total mass of acetate+acetic acid, the amount of acetate double or halves, respectively.

### Example IV

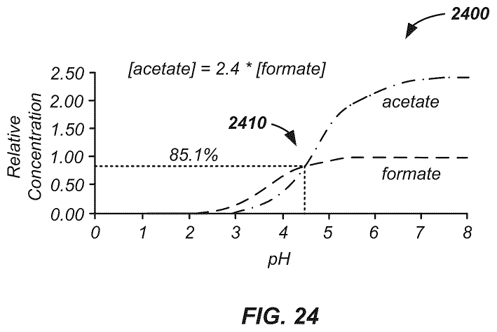

Referring now to FIG. 24, relative tuning of two conjugate bases 2400 is illustrated. Particularly, by adding 2.4 times as much acetic acid—acetate to a formulation as formic acid-formate, the acetate concentration is made equal to the formate concentration (35.5*2.4=85.1). Thus, the relative concentration ratio of formate-to-acetate is made to be one-to-one (1:1). Thus, the formate concentration is raised or lowered to alter the total amount of formate 324 available as a counterion 320 in a formulation of the tuned polymer 210. Similarly, the concentration of any conjugate base or first counterion is optionally and preferably tunable to any level, such as greater than 0, 0.1, 0.5, 1, 5, 10, 20, 30, 40, or 50 percent of the counterions 320 and/or a second counterion; less than 100, 90, 80, 70, 60, or 50 percent of the counterions 320 and/or a second counterion; and/or within ±1, 2, 5, 10, 25 or 50 percent of a fractional percentage of a given counterion to all counterions and/or to a second counterion of 10, 20, 30, 40, 50, 60, 70, 80, or 90 percent, as is further illustrated in the next example.

### Example V

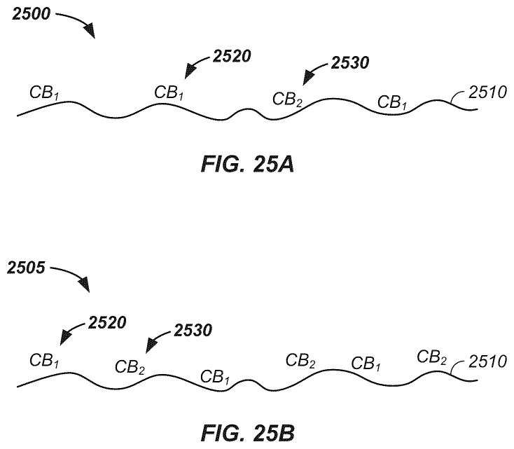

Referring now to FIG. 25A, a first counterion ratio 2500 on the polymer 200 with a backbone 2510 of atoms and/or monomers 253 is illustrated with a first conjugate base 2520 (CB₁)-to-a second conjugate base 2530 (CB₂) ratio. As illustrated the first conjugate base-to-second conjugate base ratio is three to one, 3:1. Referring now to FIG. 25B, a second counterion ratio 2505 on the polymer 200 is illustrated after tuning the conjugate base ratios/counterion ratios, as described supra. Particularly, as illustrated the first conjugate base-to-second conjugate base ratio is now tuned to one to one, 1:1, where any tuning ratio is optionally formulated, as described supra.

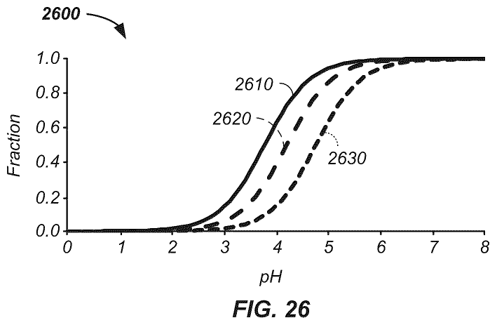

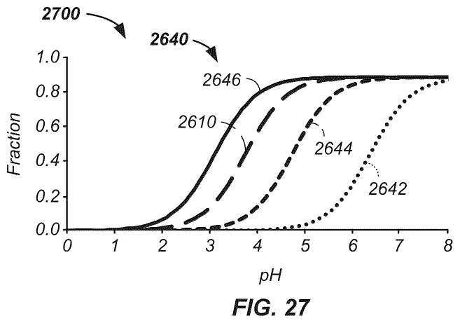

Referring now to FIG. 26, relative concentration of conjugate base tuning with three counterion types 2600 is illustrated. Here, formate fractions 2610, benzoate fractions 2620, and acetate fractions 2630 are illustrated as a function of pH, in a manner similar to that illustrated in FIGS. 21 and 22. The processes described in relation to FIGS. 20-25 are optionally repeated for three or more base pairs, such as illustrated for formate 324, benzoate, and acetate 326 herein. Thus, ratios of three base pairs are optionally controlled with a ratio of zero-to-one for the first conjugate base to zero-to-one for the second conjugate base to zero-to-one for the third conjugate base (0 to 1:0 to 1:0 to 1) at any analog step interval such as within ±0.05 of 0.1, 0.2, 0.25, or 0.5. Referring now to FIG. 27, the same process optionally uses a polyprotic acid 2700 with multiple conjugate bases, such as illustrated for the fractions of citrated 2640, such as citrate³⁻ 2642, citrate²⁻ 2644, and citrate¹⁻ 2646 with or without mixing with another conjugate base, such as the illustrated formate fraction 2610.

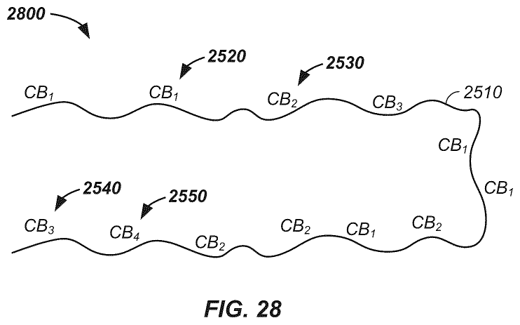

Referring now to FIG. 28, relative tuning of multiple conjugate bases/counterions 2800 is illustrated. As illustrated, the polymer 200 is associated with the first conjugate base 2520 (CB₁), the second conjugate base 2530, a third conjugate base 2540, and a fourth conjugate base 2550 at a ratio of five to four to two to one (5:4:2:1), where any ratios of conjugate bases are optionally and preferably formulated.

### Antibacterial, Antiviral, and Antifungal Testing

The inventors have determined that formate/formic acid, acetate/acetic acid, citrate/citric acid, and iodide/iodine each have antibacterial properties in combination with linear PEI, where the PEI is in solution and/or a solid film. Linear PEI with both chloride and formate counterions yielded 99.9999 percent activation using the EPA 01-1A test protocol for durability testing for both MRSA and E. coli on a glass substrate and in separate testing on stainless steel substrate with contact times of 10 minutes. Similarly, combinations of formate, acetate, and iodide with chloride yielded percent inactivation ranging from 99.4 to 99.99 using ISO 22196 and JIS 1902 test protocols for MRSA, E. coli, and fungi on substrates of (separately tested) plastic, textile, and glass with contact times of 1 minute to 10 minutes and were even effective against c. Diff with percent inactivation of 98 and 99.4 percent with contact times of 8 and 24 hours, respectively. Representative antibacterial test results are provided in Tables 1 (A-D), infra.

TABLE 1A
Linear PEI, Chloride, and Formate Antibacterial Film Testing

| | | | | Chloride | | Percent |
|---|---|---|---|---|---|---|
| Non-durability | | Contact | | and | Test | Inacti- |
| Testing | Substrate | Time | | Formate | Protocol | vation |
| Gram-positive | Plastic | 2 | min | X | IS0 22196 | 99.99 |
| MRSA | | | | | | |
| Gram-negative | Plastic | 2 | min | X | IS0 22196 | 99.99 |
| E. coli | | | | | | |
| Gram-negative | Textile | 10 | min | X | JIS 1902 | 99.4 |
| E. coli | | | | | | |
| C. diff | Glass | 24 | hours | X | IS0 22196 | 99.4 |
| Gram-negative | Stainless | 5 | min | X | PAS 2424 | 99.99 |
| E. coli. | | | | | | |
| Gram-negative | Stainless | 5 | min | X | PAS 2424 | 99.93 |
| P. Aeruginosa | | | | | | |
| Gram-positive | Stainless | 5 | min | X | PAS 2424 | 99.92 |
| MRSA | | | | | | |
| Gram-negative | Stainless | 5 | min | X | PAS 2424 | 99.94 |
| E. hire | | | | | | |
| Gram-negative | Stainless | 5 | min | X | PAS 2424 | 99.99 |
| E. coli. | | | | | | |
| Gram-negative | Glass | 10 | min | X | 01-1A | 99.999 |
| E. coli. | | | | | | |
| Gram-negative | Glass | 10 | min | X | 01-1A | 99.999 |
| P. aeruginosa | | | | | | |
| Gram-positive | Glass | 10 | min | X | 01-1A | 99.999 |
| MRSA | | | | | | |
| Gram-negative | Glass | 10 | min | X | 01-1A | 99.999 |
| E. hire | | | | | | |

TABLE 1B
Linear PEI, Chloride, and Acetate Antibacterial Film Testing

| | | | | Chloride | | Percent |
|---|---|---|---|---|---|---|
| Non-durability | | Contact | | and | Test | Inacti- |
| Testing | Substrate | Time | | Acetate | Protocol | vation |
| Gram-positive | Glass | 2 | min | X | IS0 22196 | 99.99 |
| MRSA | | | | | | |
| C. diff | Glass | 8 | hours | X | IS0 22196 | 98 |
| Gram-negative | Glass | 10 | min | X | 01-1A | 99.99 |
| E. coli. | | | | | | |
| Gram-negative | Glass | 10 | min | X | 01-1A | 99.99 |
| P. aeruginosa | | | | | | |
| Gram-positive | Glass | 10 | min | X | 01-1A | 99.99 |
| MRSA | | | | | | |

TABLE 1C
Linear PEI, Chloride, and Iodide Antibacterial Film Testing

| | | | Chloride | | Percent |
|---|---|---|---|---|---|
| Non-durability | | Contact | and | Test | Inacti- |
| Testing | Substrate | Time | Iodide | Protocol | vation |
| Gram-positive | Plastic | 1 min | X | IS0 22196 | 99.87 |
| MRSA | | | | | |
| Gram-negative | Plastic | 1 min | X | IS0 22196 | 99.79 |
| E. coli | | | | | |
| Gram-negative | Textile | 10 min | X | JIS 1902 | 99.4 |
| E. coli | | | | | |

TABLE 1D
Linear PEI, Chloride, and Formate Antibacterial Film Testing

| | | | Chloride | | |
|---|---|---|---|---|---|
| Durability | | Contact | and | Test | Percent |
| Testing | Substrate | Time | Formate | Protocol | Inactivation |
| Gram-positive | Glass | 10 min | X | 01-1A | 99.9999 |
| MRSA | | | | | |
| Gram-negative | Glass | 10 min | X | 01-1A | 99.9999 |
| E. coli | | | | | |
| Gram-positive | Stainless | 10 min | X | 01-1A | 99.9999 |
| MRSA | | | | | |
| Gram-negative | Stainless | 10 min | X | 01-1A | 99.9999 |
| E. coli | | | | | |

The inventors have determined that formate/formic acid, acetate/acetic acid, citrate/citric acid, and iodide/iodine each have antiviral properties in combination with linear PEI. Using government approved testing and/or internal tests paralleling government approved tests, linear PEI with chloride and formate counterions yielded 99.6 to 99.9 percent inactivation of enveloped viruses; linear PEI with chloride and acetate counterions yielded 99.95 to 99.99 inactivation of enveloped and non-enveloped viruses; and linear PEI with chloride and iodide counterions yielded 89.7 percent inactivation of the very difficult non-enveloped polio virus, where the tests were performed on a variety of substrates with contact times ranging from 5 to 10 minutes. Representative antiviral test results are provided in Tables 2(A-C), infra.

TABLE 2A
Linear PEI, Chloride, and Formate Antiviral Testing

| | | | | Chloride | | Percent |
|---|---|---|---|---|---|---|
| Durability | | Contact | | and | | Inacti- |
| Testing | Substrate | Time | | Formate | Test Protocol | vation |
| Vaccinia | Plastic | 5 | min | X | IS0 22702 | 99.96 |
| enveloped | | | | | | |
| Virus | | | | | | |
| Beta Corona | Stainless | 5 | min | X | IS0 22702 | 99.9 |
| enveloped | | | | | | |
| Virus | | | | | | |
| Polio Non- | Plastic | 10 | min | X | EN 14476 | 99.6 |
| enveloped | | | | | | |
| Virus | | | | | | |
| Vaccinia | Stainless | 10 | min | X | PAS 2424 | 99.96 |
| enveloped | | | | | | |
| virus | | | | | | |
| Beta Corona | Artificial | 10 | min | X | PAS 2424 | 99.9 |
| enveloped | Skin | | | | | |
| virus | | | | | | |

TABLE 2B
Linear PEI, Chloride, and Acetate Antiviral Testing

| | | | Chloride | | Percent |
|---|---|---|---|---|---|
| Durability | Sub- | Contact | and | | Inacti- |
| Testing | strate | Time | Acetate | Test Protocol | vation |
| MS2 Surrogate | Glass | 5 min | X | IS0 22702 | 99.99 |
| for Non- | | | | | |
| enveloped | | | | | |
| Virus | | | | | |
| Enveloped | Spray | 5 min | X | EXI .2.10.26.17 | 99.95 |
| H1N1 | test | | | | |
| Non-enveloped | Spray | 5 min | X | EXI .2.10.26.17 | 99.95 |
| Rhinovirus | test | | | | |

TABLE 2C
Linear PEI, Chloride, and Iodide Antiviral Testing

| | | | Chloride | | Percent |
|---|---|---|---|---|---|
| Durability | | Contact | and | | Inacti- |
| Testing | Substrate | Time | Iodide | Test Protocol | vation |
| Non-enveloped | Textile | 10 min | X | IS0 22702 | 89.7 |
| Polio | | | | | |
| Polio Non- | Stainless | 10 min | X | PAS 2424 | 89 |
| enveloped | | | | | |
| virus | | | | | |

The inventors have determined that formate/formic acid, acetate/acetic acid, citrate/citric acid, and iodide/iodine each have antifungal properties in combination with linear PEI. Using government approved testing, linear PEI with chloride and formate counterions yielded 99.6 to 99.9 percent inactivation of fungus. Referring now to FIG. 3A, representative antifungal test results are provided.

TABLE 3A
Linear PEI, Chloride, and Formate Antifungal Film Testing

| | | | Chloride | | Percent |
|---|---|---|---|---|---|
| Non-durability | | Contact | and | Test | Inacti- |
| Testing | Substrate | Time | Formate | Protocol | vation |
| Fungi | Textile | 2 min | X | JIS 1902 | 99.6 |
| Fungi | Glass | 5 min | X | IS0 22196 | 99.99 |
| Fungus | Stainless | 5 min | X | PAS 2424 | 99.94 |
| Candida auris | | | | | |

### Chemical Backbone 2510

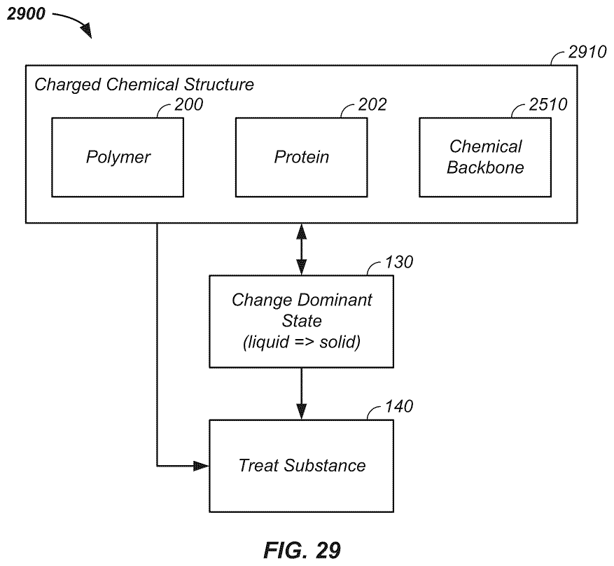

Referring now to FIGS. 29 to 36, the backbone 2510 is further described. Herein, the polymer 200 is used as an example of a chemical backbone 2510 of a charged chemical structure 2910. Stated again, the polymers 200 described herein are used to carry a plurality of charges, such as cationic charges, along a chemical backbone 2510. However, the chemical backbone 2510, of which the polymer 200 is an example, is optionally any sequence of covalently bonded atoms, such as described infra.

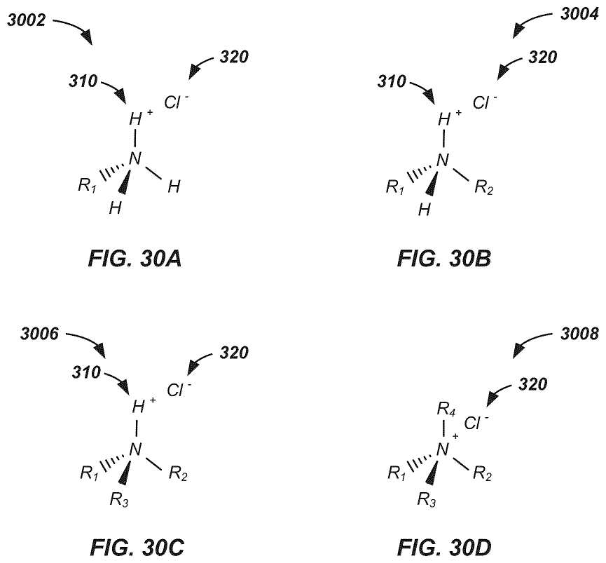

Referring now to FIG. 29, a process of treating a substance with a charged chemical structure 2900 is illustrated. For clarity of presentation and without loss of generality, a polymer 200 is used herein to represent the charged chemical structure 2910. However, the charged chemical structure is optionally a protein 202 or more generically any chemical backbone 2510. Generally, the chemical backbone 2510 is a series of covalently bonded atoms, amino acids, monomers, and/or functional groups that optionally and preferably carry multiple charges, as further described in FIGS. 30-36. As with the polymer tuning process 100 illustrated in FIG. 1, the protein 202 and/or the chemical backbone 2510 are optionally and preferably tuned, such as by any process described herein for tuning the polymer 200. As with the polymer 200, the now multi-charged/polycationic protein and/or multi-charged/polycationic chemical backbone are optionally and preferably used to treat a substance 140, such as after an optional change in dominant state 130, where the treatment yields antimicrobial 231, antibacterial 232, antiviral 233, and/or antifungal 234 properties. For clarity of presentation and without loss of generality, whenever a polymer 200, such as a tuned polymer 210 and/or a polyethylenimine 250 is referred to, reference to the more generic chemical backbone 2510 is optionally and preferably inferred.

Referring now to FIGS. 30(A-D), the charged chemical backbone 2510 optionally and preferably contains charged sites, such as a charged nitrogen. For clarity of presentation, four amine types are illustrated. Referring now to FIG. 30A, a primary amine 3002 is illustrated, where, in a positively charged state, a nitrogen is bonded to three hydrogens and one additional molecular fragment, referred to here as a first molecular fragment, R₁. Often, the first molecular fragment terminates in a carbon bound to the nitrogen, where the carbon is of any hybridization. In the positively charged state, the primary amine 3002 is illustrated as protonated 310 and is illustrated with a counterion 320. Referring now to FIG. 30B, a secondary amine 3004 is illustrated, where, in a positively charged state, the nitrogen is bonded to two hydrogens and two additional molecular fragments, referred to here as a first molecular fragment, R₁, and a second molecular fragment, R₂. Again, the second molecular fragment and/or is any molecular fragment and optionally terminates in a carbon bound to the nitrogen. Referring now to FIG. 30C, a tertiary amine 3006 is illustrated, where, in a positively charged state, the nitrogen is bonded to a single hydrogen and three additional molecular fragments, referred to here as a first molecular fragment, R₁; a second molecular fragment, R₂; and a third molecular fragment, R₃. Referring now to FIG. 30D, a quaternary amine 3008 is illustrated, where the nitrogen is bonded to four molecular fragments, designated R₁, R₂, R₃, and R₄.

Still referring to FIGS. 30(A-D), the polymers illustrated herein, such as the polyethylenimine 250, linear polyethylenimine 3300, and/or the branched polyethylenimine 3400 contain primary amines 3002, secondary amines 3004, and/or tertiary amines 3006, optionally and preferably in the absence of quaternary amines 3004, which are relatively toxic in comparison. Quaternary ammonium compounds, or quats, are a group of positively charged ions that are often used as disinfectants, surfactants, and fabric softeners. While quaternary ammonium compounds are generally considered safe for use in household and industrial settings, concentrations are environmentally limited as the quaternary ammonium compounds may be toxic at high concentrations, with long exposure times, and/or if ingested. Herein, any one or more quaternary ammonium compounds/molecules are optionally used in combination with any of the polymers 200, the polyethylenimine 250, and/or the multi-charged chemical backbones 2510 described herein.

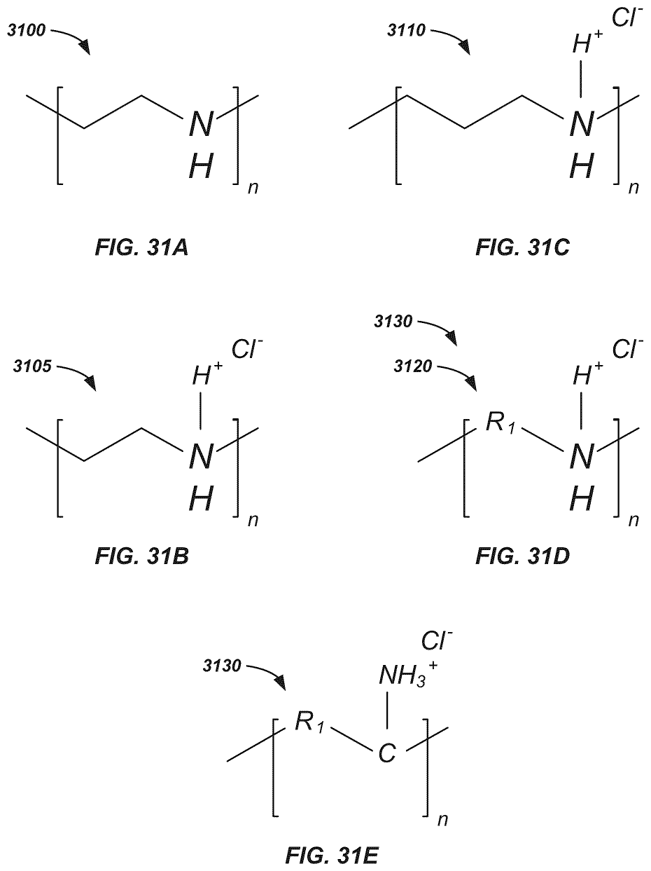

Referring now to FIG. 31A and FIG. 31B, an exemplary repeating group, such as found in the polymer 200, the polyethylenimine 250, and in some cases the chemical backbone 2510 is illustrated. Referring still to FIG. 31A and referring again to FIG. 2, the illustrated repeating monomer 253 is the repeating unit of polyethylenimine 250 in a basic form 3100. Referring now to FIG. 31B, the repeating unit of polyethylenimine 250 in an acidic form 3105 is illustrated. In polyethylenimine 250, the repeating unit includes a chain of covalently bonded atoms in a repeating sequence of carbon-carbon-nitrogen. Again, in FIG. 31B, the acidic form of the nitrogen is protonated and has a counterion and is referred to as a protonated amine and more specifically a protonated secondary amine. However, the repeating monomer is optionally of any structure. Instead of the repeating carbon-carbon-nitrogen group of the polyethylenimine illustrated in FIG. 31B, a repeating carbon-carbon-carbon-nitrogen group of polypropylenimine 3110 is illustrated in FIG. 31C. Even more generally, the repeating group is optionally of any structure bonded to the nitrogen referred to here as R₁-nitrogen, or simply a repeating unit of a nitrogen containing backbone 3120. For instance, the polyethylenimine polymer has a repeating carbon-carbon-nitrogen backbone, the polypropylenimine polymer has a repeating carbon-carbon-carbon-nitrogen backbone, and more generally the polymer 200 has a repeating nitrogen containing backbone, such as R₁-nitrogen, or R₁-nitrogen-R₂, where R₁ and R₂ are each independently any organic molecular fragment, such as of any length of carbons, and/or of any length of carbons covalently bonded to and/or interspersed with any other atoms, such as carbon-oxygen-carbon-carbon-carbon-, which is bonded to the nitrogen. Optionally, the nitrogen is not in the backbone structure, but is rather attached to the backbone structure of the repeating group, such as an —NH₂ or —NH₃ group attached to a carbon in the backbone structure. Optionally, the nitrogen is indirectly covalently bonded to the backbone structure. Herein, where the nitrogen, which is optionally protonated, is attached in any manner to the backbone structure of the repeating unit, the combined nitrogen containing fragment and the backbone fragment of the polymer is referred to as a nitrogen containing repeating backbone monomer 3130/fragment of the polymer.

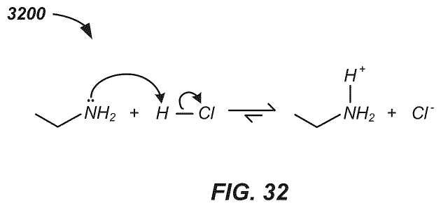

Referring now to FIG. 32, acidification of a base 3200 is illustrated. More specifically, an example is provided showing the relationship between the basic repeating unit of polyethylenimine 250 and a protonated repeating unit of the polyethylenimine 250. Generally, the base, which is a proton acceptor and/or an electron donor, interacts with an acid, such as hydrochloric acid, which is a proton donor and/or an electron acceptor to form a conjugate acid of the base and a counterion.

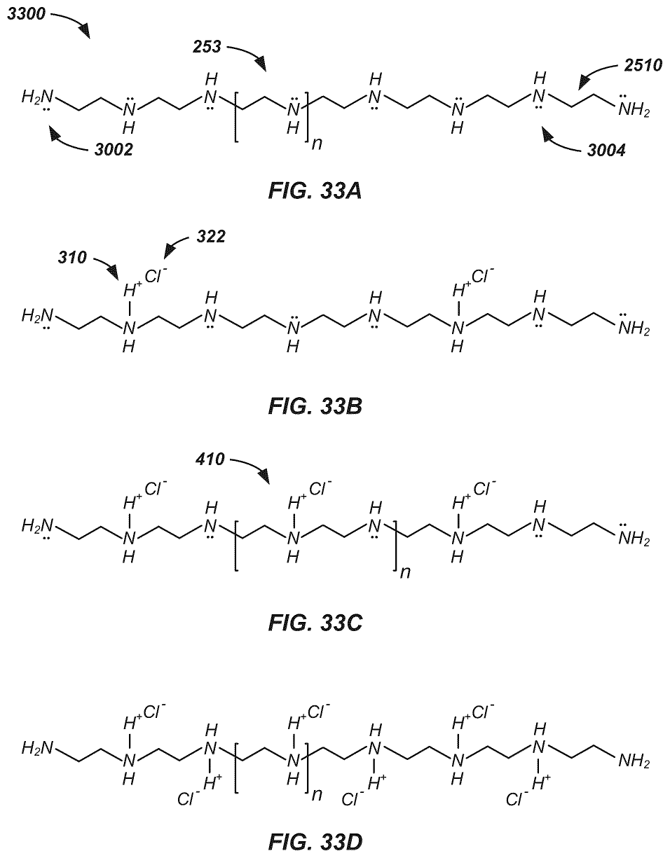

Referring now to FIGS. 33(A-D), linear polyethylenimine 3300, referred to as linear PEI, is sequentially illustrated in a base form, a slightly acidified form, a more acidified form, and an acidified form, respectively. Referring again to FIG. 33A, polyethylenimine 250 is illustrated with primary amines 3002 at the polymer termini and a multitude of secondary amines 3004, such in the repeating monomer 253. Again, the linear polyethylenimine 3300 contains a chemical backbone 2510 of repeating units of carbon-carbon-nitrogen. Referring again to FIG. 33B, the partially acidified polyethylenimine 250 is illustrated as partially protonated 310 with a counterion 320, in the illustrated case a chloride ion 322/chloride counterion. Referring again to FIG. 33C, optionally in small sections of the polyethylenimine 250, a dimer is present of one charged nitrogen monomer unit and one uncharged nitrogen monomer unit 413. Notably, the frequency of the charged nitrogen monomer is every monomer unit, every second, third, fourth monomer unit, and/or at random intervals along the length of the polyethylenimine 250. The polymer is optionally referred to a percent protonated, such as greater than 1, 2, 5, 10, 30, or 50 percent protonated and/or as less than 99, 98, 95, 90, 70, or 50 percent protonated. Referring again to FIG. 33D, a fully protonated version of the polyethylenimine 250 is illustrated. Generally, referring again to FIGS. 33(A-D), as the pH is decreased, the percentage of protonated sites on the polyethylenimine 250 increases and vice-versa.

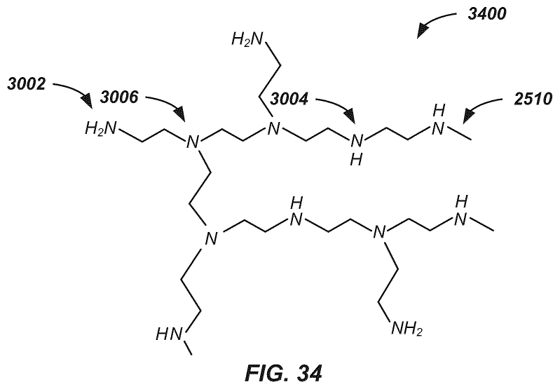

Referring now to FIG. 34, branched polyethylenimine 3400 is illustrated, which is an example of another chemical backbone 2510. The branched polyethylenimine 3400 contains the primary amines 3002 and the secondary amines 3004 of linear polyethylenimine 3300 and contains tertiary amines 3006. As illustrated, at least some of the nitrogens in the tertiary amines are covalently bonded to three carbon-carbon-nitrogen fragments. Again, the linear polyethylenimine 3300 and branched polyethylenimine 3400 contain no quaternary amines 3008, but quaternary amines 3008 are optionally added to a formulation of the polyethylenimines 250 or any polymer 200 to enhance antimicrobial 231, antibacterial 232, antiviral 233, and/or antifungal 234 properties.

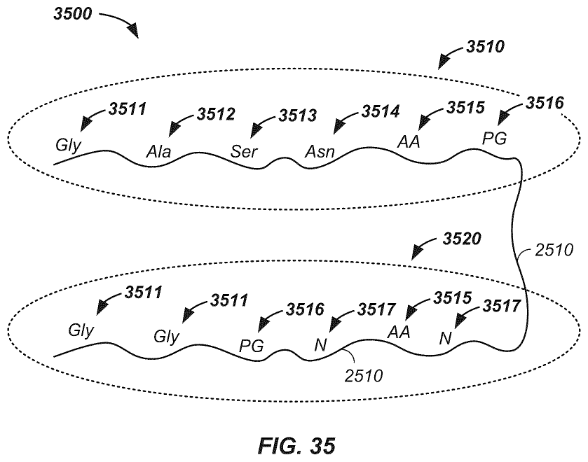

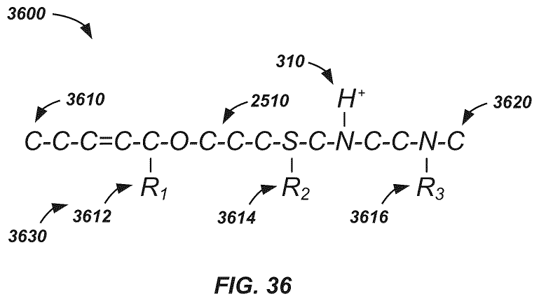

Referring now to FIG. 35 and FIG. 36, for clarity of presentation and without loss of generality, two exemplary chemical backbones 2510 are illustrated.

### Example I

Referring now to FIG. 35, a first example of a chemical backbone 3500 is illustrated with amino acids/protein sections in place of the polymer 200. As illustrated, the chemical backbone 2510 has a first optional section 3510 of a set of amino acids, which form the chemical backbone 2510 and/or are covalently bonded, directly or indirectly, to the chemical backbone. As illustrated, the amino acids include one or more of: glycine 3511, alanine 3512, serine 3513, and/or asparagine 3514, which are each protonatable, which yields a chemical backbone 2510 with multiple cationic charges, in a manner related to the multiple cationic charged of the polymer 200/tuned polymer 210. More generally, the first optional section 3510 of the chemical backbone 3500 contains and/or is bonded directly/indirectly to one or more amino acids 3515 and/or protonatable groups 3516. As illustrated, the chemical backbone 2510 has a second optional section 3520 that intermixes one or more nitrogen groups 3517, such as a secondary amine 3004 and/or a tertiary amine 3006, into the chemical backbone 2510 and/or as molecule fragments bonded directly and/or indirectly to the chemical backbone 2510. More generally, the chemical backbone 2510 includes and/or is bonded directly/indirectly with a series of one or more amino acids, protein fragments, RNA fragments, DNA fragments, and/or peptides. Generally, the chemical backbone 2510 contains/carries a plurality of protonatable sites, such as one or more secondary amines 3004; one or more tertiary amines 3006; one or more protonatable amino acids; and/or one or more protonatable sites. Generally, the chemical backbone is of any length of greater than 10, 20, 50, or 100 covalent bonds in series.

### Example II

Referring now to FIG. 36, a second example of a chemical backbone 3600 is illustrated with a series of covalent bonds in the chemical backbone 2510 from a first terminus 3610 to a second terminus 3620 of an optional number of n termini in a branched molecule, where n is a positive integer of at least two. Generally, the chemical backbone 2510 contains, directly/indirectly, any number of protonatable groups 310. Generally, the chemical backbone 2510 contains any combination of carbon, oxygen, sulfur, and nitrogen atoms covalently bonded in a series of greater than 5, 10, 15, 20, 50, 100, 500, or 1000 covalent bonds, where any of the covalent bonds are optionally double bonds. The illustrated series of covalently bonded atoms is illustrative in nature only and is non-limiting. The chemical backbone 2510 and/or any side chain/group bonded directly/indirectly to the chemical backbone 2510 contains greater than 3, 4, 5, 10, 20, 50, 100, 200, 500, 1000, 5000, 10,000, or 50,000 protonatable sites, which are tunable in any manner set forth herein for polymer tuning 210. As illustrated, the chemical backbone 2510 optionally contains a set of one or more side chains 3610, such as a first side chain 3612, a second side chain 3614, and a third side chain 3616, where any of the side chains optionally contain, are bonded to, and/or are indirectly bonded to any number of protonatable groups, which are optionally tuned by any manner set forth herein for tuning the polymer 210. Generally, a set of anions are optionally used in place and/or in combination with the set of cations/protonatable groups set forth herein.

Still yet another embodiment includes any combination and/or permutation of any of the elements described herein.

Herein, any number, such as 1, 2, 3, 4, 5, is optionally more than the number, less than the number, or within 1, 2, 5, 10, 20, or 50 percent of the number.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

In the foregoing description, the invention has been described with reference to specific exemplary embodiments; however, it will be appreciated that various modifications and changes may be made without departing from the scope of the present invention as set forth herein. The description and figures are to be regarded in an illustrative manner, rather than a restrictive one and all such modifications are intended to be included within the scope of the present invention. Accordingly, the scope of the invention should be determined by the generic embodiments described herein and their legal equivalents rather than by merely the specific examples described above. For example, the steps recited in any method or process embodiment may be executed in any order and are not limited to the explicit order presented in the specific examples. Additionally, the components and/or elements recited in any apparatus embodiment may be assembled or otherwise operationally configured in a variety of permutations to produce substantially the same result as the present invention and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A method for generating a biocide, comprising the steps of:
generating a solution of polymers comprising charged amines on nitrogen containing repeating backbone monomers, said solution comprising chloride anions as counterions to greater than fifty percent of said charged amines and a pH less than four; and subsequently:
increasing a pH of said solution by at least one pH unit; and
decreasing said chloride anion concentration of said solution by at least five percent to yield a reduced chloride anion solution,
said reduced chloride anion solution yielding said biocide, said biocide inactivating, with physical contact for greater than two minutes, both: greater than fifty percent of a bacteria and fifty percent of a virus.

2. The method of claim 1, said step of generating further comprising:
generating said solution of polymers with a repeating monomer, said repeating monomer comprising a carbon-carbon-nitrogen backbone section.

3. The method of claim 1, said step of generating further comprising:
generating said solution of polymers with polyethylenimine polymers, said charged amines comprising charged secondary amines.

4. The method of claim 3, said step of generating further comprising the step of:
yielding chloride counterions to greater than eighty percent of said charged secondary amines.

5. The method of claim 3, further comprising the step of:
raising said pH of said solution to greater than 6 to form a solid salt.

6. The method of claim 5, said step of raising further comprising the step of:
combining an organic acid with said solution containing said polyethylenimine polymers to raise said pH of said solution to greater than 6.

7. The method of claim 5, said step decreasing further comprising the steps of:
removing, after said step of raising said pH of said solution to greater than 6, greater than ten percent of said solid salt on said biocide; and
reducing pH, after said step of removing, of said biocide by at least one-half of a pH unit to form a reduced salt solution comprising said biocide.

8. The method of claim 1, further comprising the steps of:
raising said pH of said solution to greater than 6;
said step decreasing further comprising the step of removing, after said step of raising said pH of said solution to greater than 6, greater than ten percent of chloride anions in said solution; and
reducing pH of said solution by at least one-half of a pH unit.

9. The method of claim 1, further comprising the step of:
adding anion exchange material to said solution, said anion exchange material simultaneously performing said steps of: (1) increasing said pH of said solution by at least one pH unit and (2) decreasing said chloride anion concentration of said solution by at least five percent.

10. The method of claim 9, further comprising the step of:
forming a solid film from said solution through evaporation of at least eighty percent of an organic solvent in said solution, said film comprising a total cationic charge in a range of 0.6 to 5 coulombs.

11. The method of claim 4, further comprising the step of:
reducing said chloride anion concentration by at least thirty percent.

12. The method of claim 4, further comprising the step of:
reducing a concentration of said chloride counterions to, on average, association with less than seven of every eight of said charged secondary amines.

13. The method of claim 1, further comprising the steps of:
forming a solid film with said solution; and
maintaining said inactivation after greater than three cycles of rubbing, for greater than ten seconds, the solid film with a pressure exceeding four pounds of pressure.

14. The method of claim 1, further comprising the step of:
performing both said step of increasing said pH and said step of decreasing said chloride anion concentration simultaneously.

15. The method of claim 14, said step of performing further comprising the step of:
exchanging ions from an anion exchange material for ions from said solution.

16. The method of claim 3, further comprising the step of:
removing counterions to said charged secondary amines of said polyethylenimine from said solution with said anion exchange material.

17. The method of claim 16, said step of removing counterions further comprising the step of:
removing chloride anions from said solution with said anion exchange material.

18. The method of claim 1, said step of generating further comprising a step of:
combining with said polymers a source of protons other than hydrochloric acid.

19. The method of claim 4, said step of generating further comprising the step of:
adding a strong acid to said polyethylenimine polymers, said strong acid comprising at least one of:
hydrobromic acid;
hydroiodic acid;
nitric acid;
perchloric acid;
sulfuric acid; and
chloric acid.

20. The method of claim 19, further comprising the step of:
simultaneously raising pH of said solution and decreasing anions in said solution through addition of an anion exchange material to said solution.
